(19) United States
(12) Patent Application Publication
(10) Pub. No.: US 2024/0041349 A1
(43) Pub. Date: Feb. 8, 2024

(54) Gait Analysis Devices, Methods, and Systems

(71) Applicant: The Trustees of Columbia University in the City of New York, New York, NY (US)

(72) Inventors: Sunil K. AGRAWAL, Newark, DE (US); Jesus Antonio PRADO DE LA MORA, New York, NY (US)

(73) Assignee: The Trustees of Columbia University in the City of New York, New York, NY (US)

(21) Appl. No.: 18/379,487

(22) Filed: Oct. 12, 2023

Related U.S. Application Data
(63) Continuation of application No. 16/556,961, filed on Aug. 30, 2019.
Application No. 16/556,961 is a continuation-in-part of application No. 15/305,145, filed on Oct. 19, 2016, now abandoned.
(60) Provisional application No. 62/731,333, filed on Sep. 14, 2018.
(60) Provisional application No. 61/982,832, filed on Apr. 22, 2014.

(51) Int. Cl. A61B 5/103; A61B 5/00

(52) U.S. Cl. CPC A61B 5/1038; A61B 5/6807; A61B 5/7405; A61B 5/7455; A61B 5/7264; A61B 5/4082; A61B 2562/0219; A61B 2562/0247; A61B 2562/046; A61B 2562/0204

(57) ABSTRACT

A quantitative gait training and/or analysis system employs instrumented footwear and an independent processing module. The instrumented footwear may have sensors that permit the extraction of gait kinematics in real time and provide feedback from it. Embodiments employing calibration-based estimation of kinematic gait parameters are described. An artificial neural network identifies gait stance phases in real-time.

### CROSS-REFERENCE TO RELATED APPLICATIONS

The present application is a continuation of U.S. patent application Ser. No. 16/556,961, filed Aug. 30, 2019, which claims the benefit of U.S. Provisional Application No. 62/731,333, filed Sep. 14, 2018, and which is also a continuation-in-part of U.S. patent application Ser. No. 15/305,145, filed Oct. 19, 2016, which is a national stage filing of International Application No. PCT/US2015/027007, filed Apr. 22, 2015, which claims the benefit of U.S. Provisional Application No. 61/982,832, filed Apr. 22, 2014, each of which are hereby incorporated by reference herein in their entireties.

### FIELD

The present disclosure relates generally to systems, methods, and devices for gait analysis and training, and, more particularly, to a wearable, autonomous apparatus for quantitative analysis of a subject's gait and/or providing feedback for gait training of the subject.

### BACKGROUND

Pathological gait (e.g., Parkinsonian gait) is clinically characterized using physician observation and camera-based motion-capture systems. Camera-based gait analysis may provide a quantitative picture of gait disorders. However, camera-based motion capture systems are expensive and are not available at many clinics. Auditory and tactile cueing (e.g., metronome beats and tapping of different parts of the body) are often used by physiotherapists to regulate patients' gait and posture. However, this approach requires the practitioner to closely follow the patient and does not allow patients to exercise on their own, outside the laboratory setting.

### SUMMARY

Systems, methods, and devices for gait training and/or analysis are disclosed herein. An autonomous system is worn by a subject, thereby allowing for analysis of the subject's gait and offering sensory feedback to the subject in real-time. One or more footwear units or modules are worn by a subject. Sensors coupled to or embedded within the footwear unit measure, for example, underfoot pressure and feet kinematics as the subject walks. A processing unit, also worn by the subject, processes data from the sensors and generates appropriate auditory and vibrotactile feedback via the footwear unit units in response to these input data. Embodiments of the disclosed subject matter may be especially advantageous for subjects that have reduced functionality in their lower limbs, reduced balance, or reduced somatosensory functions. Feedback provided by the system may help regulate wearer's gait, improve balance, and reduce the risk of falls, among other things.

In embodiments, a gait training and analysis system may be worn by a subject. The system may include a pair of footwear modules, a processing module, and signal cables such as audio cables. The footwear units may be constructed to be worn on the feet of the subject. Each footwear module may comprise a sole portion, a heel portion, a speaker, and a wireless communication module. The sole portion may have a plurality of piezo-resistive pressure sensors and a plurality of vibrotactile transducers. Each piezo-resistive sensor may be configured to generate a sensor signal responsively to pressure applied to the sole portion, and each vibrotactile transducer may be configured to generate vibration responsively to one or more feedback signals. The heel portion may have a multi-degree of freedom inertial sensor. The speaker may be configured to generate audible sound in response to the one or more feedback signals. The wireless communication module may be configured to wirelessly transmit each sensor signal. The processing module may be constructed to be worn as a belt by the subject. The processing module may be configured to process each sensor signal received from the wireless communication module and to generate the one or more feedback signals responsively thereto. The signal cables may connect each footwear module to the processing module and may be configured to convey the one or more feedback signals from the processing module to the vibrotactile transducers and speakers of the footwear unit.

In embodiments, a system for synthesizing continuous audio-tactile feedback in real-time may comprise one or more sensors and a computer processor. The one or more sensors may be configured to be attached to a footwear unit device of a subject to measure pressure under the foot and/or kinematic data of the foot. The computer processor may be configured to be attached to the subject to receive data from the one or more sensors and to generate audio-tactile signals based on the received sensor data. The generated audio-tactile signal may be transmitted to one or more vibrotactile transducers and loudspeakers included in the footwear unit.

In embodiments, a method for real-time synthesis of continuous audio-tactile feedback may comprise measuring pressure and/or kinematic data of a foot of a subject, sending the pressure and/or kinematic data to a computer processor attached to a body part of the subject to generate audio-tactile feedback signal based on the measured pressure and/or kinematic data, and sending the audio-tactile feedback signal to vibrotactile sensors attached to the foot of the subject.

In embodiments, a system may comprise one or more footwear modules, a feedback module, and a wearable processing module. Each footwear module may comprise one or more pressure sensors and one or more inertial sensors. The feedback module may be configured to provide a wearer of the footwear unit with at least one of auditory and tactile feedback. The wearable processing module may be configured to receive signals from the pressure and inertial sensors and to provide one or more command signals to the feedback module to generate the at least one of auditory and tactile feedback responsively to the received sensor signals.

In embodiments, a method for gait analysis and/or training may comprise generating auditory feedback via one or more speakers and/or tactile feedback via one or more vibrotactile transducers of the footwear unit. The generating may be responsive to signals from pressure and inertial sensors of the footwear unit indicative of one or more gait parameters.

Objects and advantages of embodiments of the disclosed subject matter will become apparent from the following description when considered in conjunction with the accompanying drawings.

### BRIEF DESCRIPTION OF DRAWINGS

Embodiments will hereinafter be described with reference to the accompanying drawings, which have not necessarily been drawn to scale. Where applicable, some features may not be illustrated to assist in the illustration and description of underlying features. Throughout the figures, like reference numerals denote like elements.

FIG. 1 is schematic diagram illustrating components of a system for gait analysis and training, according to one or more embodiments of the disclosed subject matter.

FIG. 2A is a schematic diagram illustrating components of a footwear unit of a system for gait analysis and training, according to one or more embodiments of the disclosed subject matter.

FIGS. 2B and 2C are side and bottom views of an exemplary footwear module for gait analysis and training, according to one or more embodiments of the disclosed subject matter.

FIG. 3A is a schematic diagram illustrating further components of a system for gait analysis and training, according to one or more embodiments of the disclosed subject matter.

FIG. 3B is an image of a bottom of an exemplary footwear module, according to one or more embodiments of the disclosed subject matter.

FIG. 3C is an image of an exemplary system for gait analysis and training worn by a subject, according to one or more embodiments of the disclosed subject matter.

FIG. 3D is an image of a side of an exemplary footwear module, according to one or more embodiments of the disclosed subject matter.

FIG. 4 shows graphs of a feedback generation process for a step using the system for gait analysis and training, including a time derivative of normalized pressure values underneath the heel and toe (top graph), 1-norm of dynamic acceleration (second graph), exciter signal scaled in amplitude (third graph), and a synthesized signal simulating snow (bottom graph).

FIG. 5 illustrates an experimental protocol for evaluating the system for gait analysis and training.

FIG. 6 is a graph of average stride time measured by the system for gait analysis and training for different bases.

FIG. 7 is a graph of normalized impact force at initial contact measured by the system for gait analysis and training for different bases.

FIG. 8 is a graph of average step length measured by the system for gait analysis and training for different bases.

FIG. 9 is a graph of average swing period measured by the system for gait analysis and training for different bases.

FIG. 10A is a schematic diagram illustrating further components of another system for gait analysis and training, according to one or more embodiments of the disclosed subject matter.

FIG. 10B is an image of the system of FIG. 10A worn by a subject, according to one or more embodiments of the disclosed subject matter.

FIG. 10C is an image of a bottom of an exemplary footwear module, according to one or more embodiments of the disclosed subject matter.

FIG. 10D is an image of a side of an exemplary footwear module, according to one or more embodiments of the disclosed subject matter.

FIG. 11 is an image illustrating the positions of reflective markers for calibration of a system for gait analysis and training, according to one or more embodiments of the disclosed subject matter.

FIGS. 12a through 12v shows graphs of correlation, frequency distribution of measurement error, and Bland-Altman plots for the system for gait analysis and training, according to one or more embodiments of the disclosed subject matter.

FIGS. 13A through 14B illustrate different arrangements for the footwear units and processing module worn by a subject, according to one or more embodiments of the disclosed subject matter.

FIGS. 15 and 16 show calibration procedures for generating subject-specific and subject-generic production estimation models for kinematic parameters which may be used for generation of real time feedback, according to embodiments of the disclosed subject matter.

FIG. 17 shows a production method for generation of real time feedback responsively to a generic or subject-specific model, according to embodiments of the disclosed subject matter.

FIG. 18A shows a subject wearing an instrumented shoe according to embodiments of the disclosed subject matter.

FIG. 18B shows a printed circuit board with an inertial measurement unit and microprocessor used in conjunction with the instrumented shoe of FIG. 18A and other embodiments, according to embodiments of the disclosed subject matter.

FIG. 18C shows an instrumented insole of the shoe according to embodiments of the disclosed subject matter.

FIG. 19A shows a graphical representation of a normal gait cycle and how the events are defined by heel strikes and toe off according to embodiments of the disclosed subject matter.

FIG. 19B shows an example of a binary function of the gait phases according to embodiments of the disclosed subject matter.

FIG. 20 shows a network architecture for a segmentation model phases according to embodiments of the disclosed subject matter.

FIGS. 21A through 21F show error distributions of the identification errors for heel strikes with respect to the reference system according to embodiments of the disclosed subject matter.

FIGS. 22A through 22F show a network architecture for a segmentation model according to embodiments of the disclosed subject matter.

FIG. 23 shows sensor error due to variability in the walking characteristics of subjects according to embodiments of the disclosed subject matter.

FIG. 24 shows the different phases and events in a normal gait cycle.

FIG. 25 shows a data sample of 5 seconds collected by the DeepSole system.

FIGS. 26A and 26B show a graphical overview of the neural network, an encoder-decoder RNN that maps the input into gait phases.

FIGS. 27A, 27B and 27C illustrate the heel strike detection algorithm. Red line is prediction of gait phase from deep model.

FIG. 28A shows the prediction samples from deep model with fully connected encoder and decoder.

FIG. 28B shows the prediction samples from deep model with convolutional encoder and decoder.

FIG. 29A show average stance time and ratio per test. Statistical significance is shown with lines.

FIG. 29B shows average stance time and ratio per test.

### DETAILED DESCRIPTION

In one or more embodiments of the disclosed subject matter, a gait analysis and training system may provide clinicians, researchers, athletic instructors, parents and other caretakers and other individuals with detailed, quantitative information about gait at a fraction of the cost, complexity, and other drawbacks of camera-based motion capture systems. Systems may capture and record time-resolved multiple parameters and transmit reduced or raw data to a computer that further synthesizes it to classify abnormalities or diagnose conditions. For example a subject person's propensity for falling may be indicated by certain characteristics of their gait such as a wide stance during normal walking, a compensatory pattern that may be an indicator of fall-risk.

Additionally, embodiments of the disclosed gait analysis and training system may provide subjects with auditory and/or vibrotactile feedback that is automatically generated by software in real-time, with the aim of regulating/correcting their movements. The gait analysis and training system may be a wearable gait analysis and sensory feedback device targeted for subjects with reduced functionality in their lower limbs, reduced balance, or reduced somatosensory function (e.g., elderly population and PD patients). As the subject walks, the system may measure underfoot pressure, ankle motion, feet movement and generate data that may correspond to motion dynamics and responsively to these data, generate preselected auditory and vibrotactile feedback with the aim of helping the wearer adjust gait patterns or recover and thereby reduce the risk of falls or other biomechanical risks.

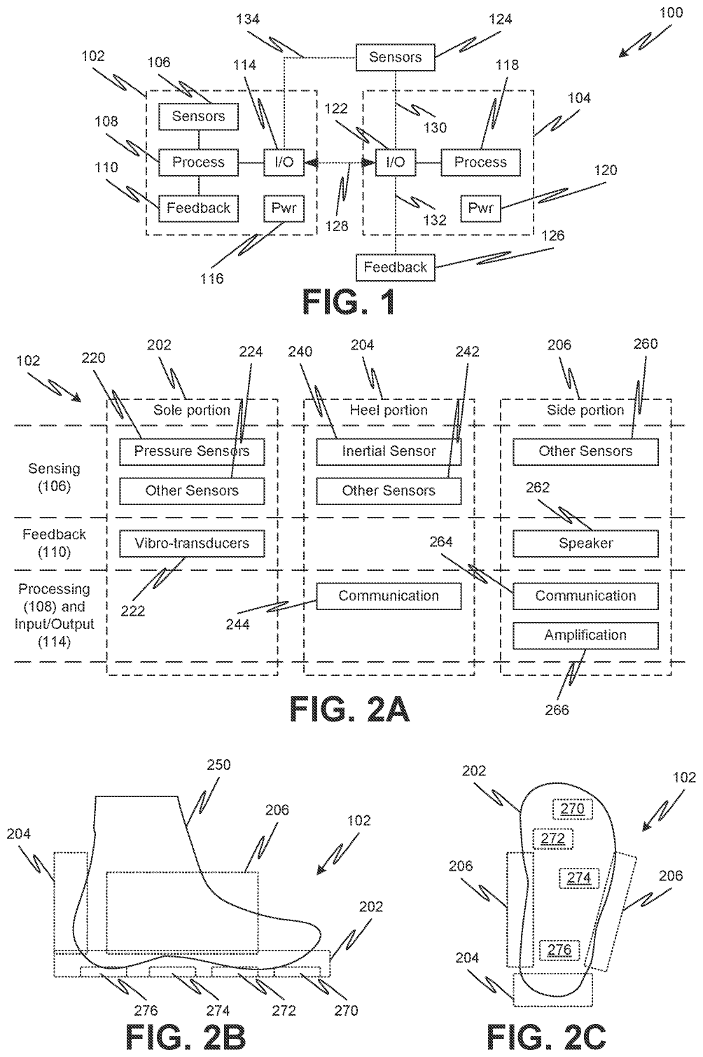

Referring to FIG. 1, a gait analysis and training system 100 may include one or more footwear modules 102 and a wearable processing module 104. The footwear unit 102 may include one or more sensors 106 that measure characteristics of the subject's gait as the subject walks, including underfoot pressure, acceleration, or other foot kinematics. The system may also include one or more remote sensors 124 disposed separate from the footwear unit 102, for example, on the shank or belt of the subject. Sensor signals from the remote sensors 124 may be communicated to the closest footwear module 102, for example, via a wired or wireless connection 134 for transmission to the remote processor 118 together with data from sensors 106 via connection 128. Alternatively, sensor signals from the remote sensors 124 may be communicated directly to the remote processor 118, for example, by wireless connection 130.

An on-board processing unit 108 may receive signals from the one or more sensors 106, 124 and prepare data responsively to the sensor signals for transmission to a remote processor 118 of the wearable processing module 104, for example, via transmission 128 between communication module 114 in the footwear unit 102 and a corresponding communication module 122 in the wearable processing module 104. The on-board processing unit 108 may include, for example, an analog to digital converter or microcontroller. For example, the transmission 128 of sensor data may be via wireless transmission

The remote processor 118 of the wearable processing module 104 may receive the sensor data and determine one or more gait parameters responsively thereto. The remote processor 118 may further provide feedback, such as vibratory or audio feedback, based on the sensor data and determined gait parameters, for example, to help the subject learn proper gait. For example, the feedback may be provided via one or more transducers 110 in the footwear unit, such as vibrotactile transducers or speakers. The transmission 128 of feedback signals from the processor 118 to the feedback transducers 110 may be via a wired connection, such as audio cables. Alternatively or additionally, the feedback may be provided via one or more remote feedback modules 126 via a wired or wireless connection 132. For example, the remote feedback module 126 may provide audio feedback via headphones worn by the subject, audio feedback via a speaker worn by the subject, tactile feedback via transducers mounted on the body of the subject remote from the foot, or visual feedback via one or more flashing lights.

The wearable processing module 104 may include an independent power supply 120, such as a battery, that provides electrical power to the components of the processing module 104, e.g., the remote processor 118 and the communication module 122. In addition, each footwear module 102 may include an independent power supply 116, such as a battery, that provides electrical power to the components of the footwear unit 102, e.g., the sensors 106, the on-board processing unit 108, the feedback transducers 110, and the communication module 114. Alternatively or additionally, the power supply 120 of the wearable processing module 104 may supply power to both the processing module 104 and the footwear units 102, for example, via one or more cables connecting the processing module 104 to each footwear module 102.

Each footwear module 102 may include at least a sole portion 202, a heel portion 204, and one or more side portions 206, as illustrated in FIGS. 2A-2C. For example, each portion of the footwear unit 102 may include sensing portions 106, feedback portions 110, and processing 108 or communication 114 portions. The sole portion 202 may include one or more pressure sensors 220 as part of sensing portion 106. Optionally, the sole portion 202 may further include one or more other sensors 224, such as an inertial measurement unit. The sole portion 202 may further include one or more vibrotactile transducers 222 as part of the feedback portion 110. The heel portion 204 of the footwear unit 102 may include one or more inertial sensors 240, such as an inertial measurement unit. Optionally, the heel portion 204 may further include one or more other sensors 242, such as an accelerometer. The heel portion 204 may further include a communication module 244, for example, a wireless communication module to transmit data from sensing portions 106 of the heel portion 204 and/or the sole portion 202. The side portions 206 may optionally include one or more other sensors, such as an ultrasonic base sensor, as part of sensing portion 106. The side portions 206 may further include a speaker 262 as part of the feedback portion 110 and a communication module 264, for example, a wired communication module to transmit feedback signals from a remote processor to the speaker 262 and/or the vibrotactile transducers 222 of the sole portion. The side portions 206 may also include an amplification module 266 to amplify the feedback signals from the remote processor.

As illustrated in FIGS. 2B-2C, feedback components and sensing devices in the sole portion 202 of the footwear unit 102 may be grouped together at various regions 270-276 along the bottom of the foot 250. For example, each region 270-276 may include at least one feedback transducer (e.g., a vibro-transducer) and at least one pressure sensor (e.g., a piezo-resistive sensor). Feedback/sensing region 270 may be disposed under the hallux distal phalanx. Feedback/sensing region 272 may be disposed under the first metatarsal head. Feedback/sensing region 274 may be disposed under the middle lateral arch and/or the fourth metatarsal head. Feedback/sensing region 276 may be disposed under the calcaneous.

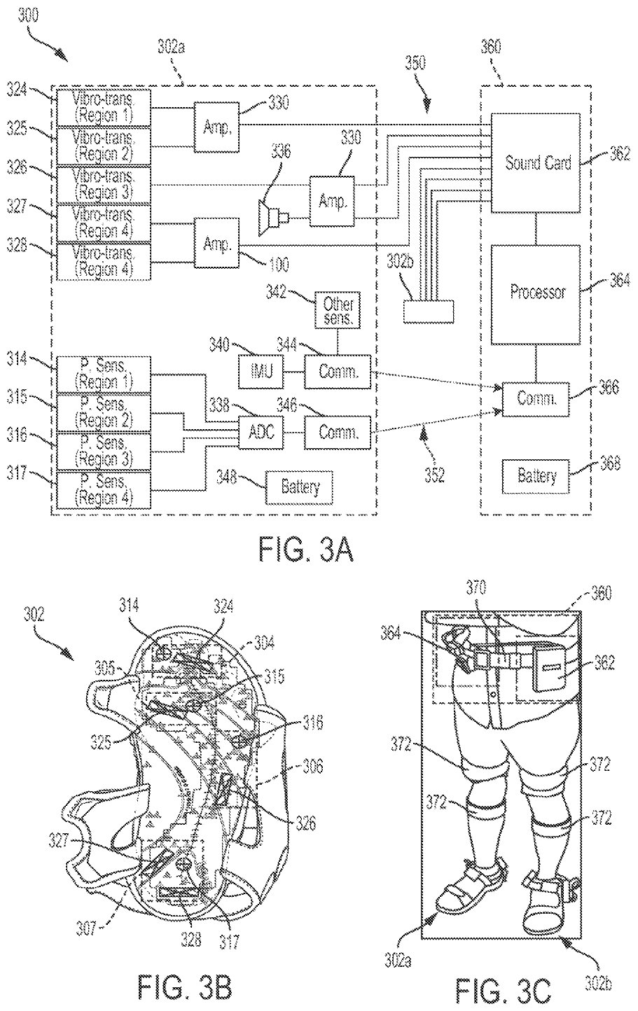

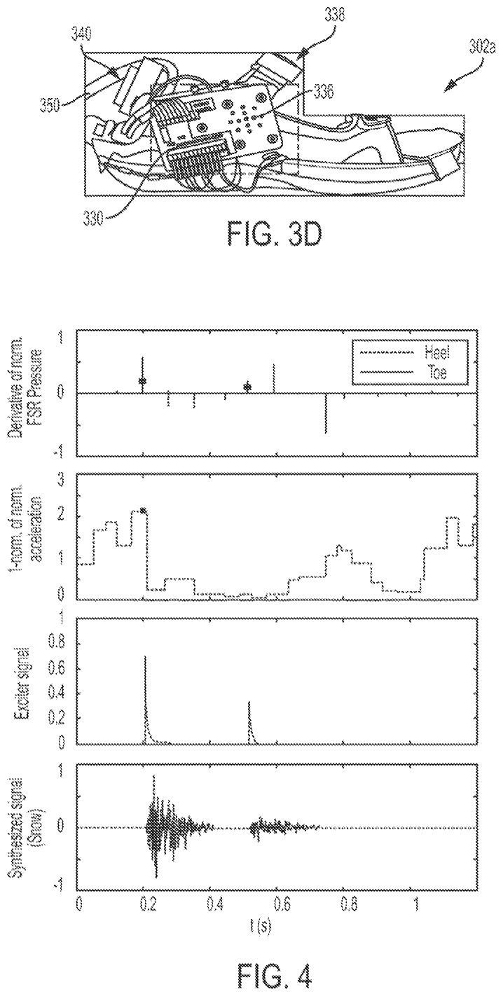

Referring to FIGS. 3A-3D, a system 300 for gait training and analysis is shown. The system 300 may include two footwear units 302a, 302b and a processing module 360 attached to the belt 370 of the subject. Each footwear unit 302a, 302b measures pressure under the foot and kinematic data of the foot. The data is sent wirelessly (e.g., via wireless connections 352) to a portable single-board computer 364 attached to the belt 370, where the audio-tactile feedback is generated in real-time and converted to analog signals by a sound card 362. Audio cables 350 (e.g., stereo audio cables similar to those used in headphones) carry the analog signals from the processing module 360 to each footwear unit 302a, 302b, where they are amplified (e.g., by one or more amplifiers 330) and fed to vibrotactile transducers 324-328 (e.g., having a nominal bandwidth of 90-1000 Hz) embedded in the sole and to one or more speakers 336 of the footwear unit 302a, 302b.

For example, the audio-tactile feedback may be converted into eight analog signals, four per leg. The vibrotactile transducers 324-328 may be placed where the density of the cutaneous mechanoreceptors in the foot sole is highest, so as to maximize the effectiveness of the vibrotactile rendering. The two anterior actuators (hallux actuator 324 and 1st metatarsal head actuator 325) may be controlled by the same first feedback signal, while the two posterior actuators (calcaneous anterior aspect actuator 327 and calcaneous posterior aspect actuator 328) may be controlled by the same third feedback signal. The other feedback components, i.e., the mid lateral arch actuator 326 and the speaker 336 may be controlled by second and fourth feedback signals, respectively.

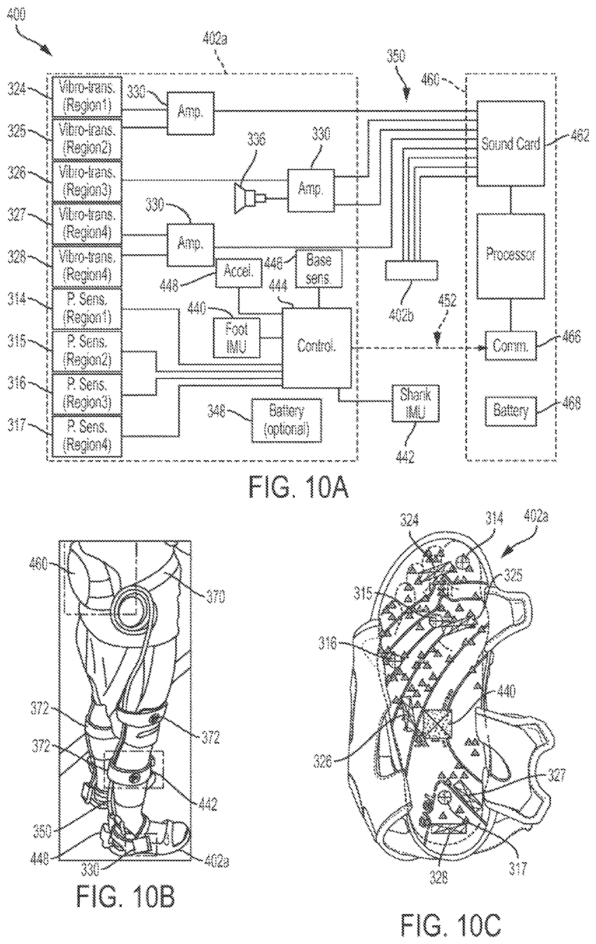

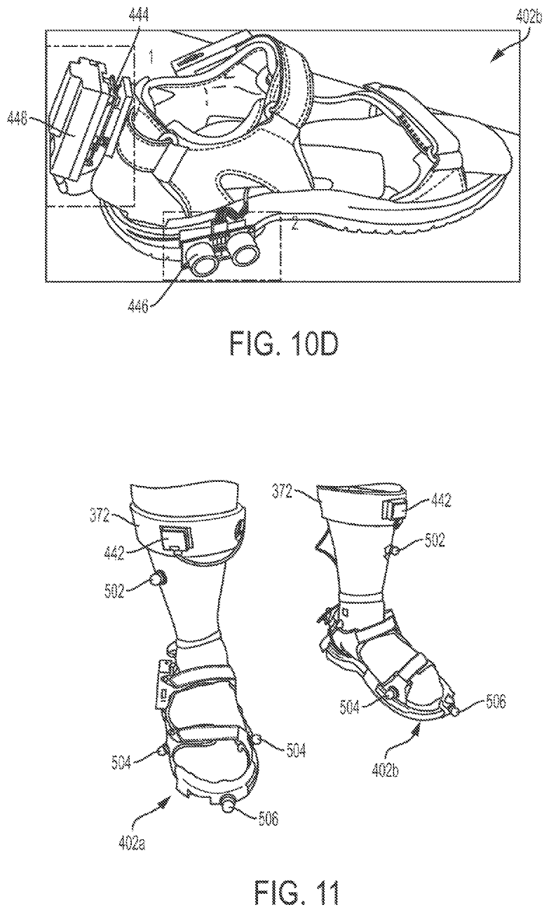

Piezo-resistive force sensors 314-317 are attached to or embedded in the sole of each footwear unit 302a, 302b. During walking, these signals peak in sequence as the center of pressure in the foot moves from the heel to the toe, thus allowing identification of the sub-phases of stance. The signals are digitized, for example, by an analog-to-digital converter 338 (ADC) and sent to processing module 360 through a first wireless module 346 (e.g., an Xbee or Bluetooth module). A multi-degree-of-freedom (DOF) inertial measurement unit 340 (IMU), for example, a 9-DOF IMU, may be mounted at various locations of the footwear unit 302a, 302b. The IMU location under the arch of the foot (See FIG. 10C and discussion thereof) thereby more remote from the heel reduces shock noise caused by heel strike and has been found to be preferable. Estimated linear acceleration of the heel and yaw-pitch-roll angles may be sent to the processing module 360 via a second wireless module 344 (e.g., an Xbee or Bluetooth module) or via the same wireless module 346 as the data from the pressure sensors 314-317.

The single-board computer 364 that attaches to the subject's belt 370 may be powered by a battery 368 (e.g., a lithium ion polymer (LiPo) battery) that fits on the top of the computer's enclosure. A real-time dataflow programming environment running in the computer 364 manages the audio-tactile footstep synthesis engine and also performs data-logging of pressure data and kinematic data on a memory device, for example, a microSD card. Modification of the feedback parameters may be accomplished by sending string commands to the computer 364 wirelessly or via an optional wired input.

The multi-channel sound card 362 of the processing module 360 may attach to the belt 370 separate from the computer 364, as illustrated in FIG. 3C, or together with the computer 364. The sound card 362 may convey audio data stream into independent analog channels. For example, two pairs of stereo cables 350 carry these audio signals to amplifiers 330 (e.g., three two-channel audio amplifier boards with 3 W per channel), which may be mounted on the lateral-posterior side of the sandals, as illustrated in FIG. 3D. The stereo cables may be bundled inside thin PET cable-sleeve that attaches to the wearer's thighs and shanks, for example using leg mounting straps 372. The cable sleeve routed through the legs does not noticeably restrict the wearer's motion.

The subject wears the footwear units 302a, 302b and the processing module 360 as the subject would do with normal shoes and a normal belt. The subject, then, connects the stereo cables 350 to the portable sound card 362 attached to a belt 370, and secures the cables to the legs with straps 372, one for each leg segment. Finally, the subject turns on the amplifiers 330 and the computer 364. The software may be programmed to start automatically, and the system 300 may operate independently, powered by on-board battery packs 348, 368. However, the subject (or a caregiver/experimenter) may change the parameters that regulate the feedback at any time, by logging into computer 364, via a wired or wireless connection through an external computer or a smartphone.

Feedback output from the vibrotactile transducers 324-328 and speaker 336 is concurrently modulated by signals from the pressure sensors 314-317 and by the motion of the foot, as estimated by the on-board inertial sensors 340 and/or other sensors 342. This allows, for example, the system 300 to generate different sounds/vibrations via the vibrotactile transducers 324-328 and speaker 336 as the subject's gait pattern changes, or as the intensity of the impact with the ground varies. Additionally, IMU sensors 340 allow estimation of the orientation and of the position of the foot in real time, which may be utilized for on-line and off-line gait analysis. Thus, embodiments of the disclosed subject matter are capable of providing multimodal feedback autonomously, i.e., without being tethered to an external host computer. All the logic and the power required for synthesizing continuous audio-tactile feedback in real-time are carried by the subject along with the power required to activate the vibrotactile actuators.

Referring to FIGS. 3A-3B, each footwear module 302 may include at least four regions 304-307 with at least one sensing component and at least one feedback component therein. For example, a first region 304 under the hallux distal phalanx of the foot includes a first piezo-resistive sensor 314 and a first vibro-transducer 324, a second region 305 under the first metatarsal head of the foot includes a second piezo-resistive sensor 315 and a second vibro-transducer 325, a third region 306 extending under the mid lateral arch and the fourth metatarsal head of the foot includes a third piezo-resistive sensor 316 and a third vibro-transducer 326, and a fourth region 307 under the calcaneous includes a fourth piezo-resistive sensor 317, a fourth vibro-transducer 327, and a fifth vibro-transducer 328. The five vibrotactile transducers 324-328 may be embedded in the sole of the footwear unit 302. The location of the transducers 324-328 may be optimized to match the sole areas where the density of mechanoreceptors is higher.

As discussed above, the gait training and analysis system 300 may utilize a hybrid wireless-wired architecture. Sensor data is sent wirelessly to the processing module 360, e.g., via wireless connection 352, whereas the feedback outputs are sent from the processing module 360 to each footwear module 302a, 302b through wired connections 350 that run along each leg. The wireless connection on the sensor side makes the system modular, meaning that additional sensors modules (e.g., additional IMUs for the upper and lower extremities) may be easily added to the system without modifying the software/hardware architecture. The wired connection at the actuators side, instead, reduces latency in generating the desired feedback.

Advantages for the subject of system 300 include, but are not limited to, regulation of the gait cycle, improvement in balance, and reduction of the risk of falls for subjects who have reduced functionality in their lower extremities, such as elderly people and subjects affected by Parkinson's disease. The cyclical coordination of joint angles, which controls the gait patterns, reflect function of subcortical circuits known as locomotor central pattern generators, which are intrinsically and biologically rhythmical. External rhythms help entrain these internal motor rhythms via close neural connections between auditory and motor areas, producing enhanced time stability, which favors spatial control of movements. Underfoot subsensory stimuli via the vibrotactile transducers 324-328 may improve somatosensory function and may produce immediate reduction of postural sway. By carrying onboard all the logic and power required for synthesizing continuous audio-tactile feedback in real-time, embodiments of the disclosed system may allow subjects to exercise on their own, e.g., at home.

The auditory and plantar vibrotactile feedback, which is rendered by a footsteps synthesis engine, may simulate foot interactions with different types of surface materials. This engine was extensively validated by means of several interactive audio-tactile experiments and is based on a number of physical models that simulate impacts, friction, crumpling events, and particle interactions. All physical models may be controlled by an exciter signal simulating the impact force of the foot onto the floor, which is normalized in the range [0, 1] and sampled at 44100 Hz. Real-time control of the engine may be achieved by generating the exciter signal of each foot based on the data of the inertial sensor 340 and of the two piezo-resistive sensors placed underneath the calcaneous 317 and the head of 1^{st} metatarsal 315. Based on the estimated orientation of the foot, the gravity component of the acceleration is subtracted from the raw acceleration. The resulting “dynamic” acceleration and the pressure values are normalized to the ranges [−1, 1] and [0, 1], respectively. Thus, the feedback intensity may be based on the ground reaction forces at initial contact obtained from inertial sensors mounted at the back of the footwear units.

The exciter corresponding to a single step is modulated by the contribution of both the heel and the forefoot strikes. The two contributions consist of ad-hoc-built signals that differ in amplitude, attack, and duration. This allows simulation of the most general case of a step, where the impact force is larger at the heel strike than at forefoot strike. These signals are triggered at the rise of the two pressure signals during a footfall as illustrated in FIG. 4, when the first derivative of each normalized pressure value becomes larger than a predefined threshold. In addition, in order to render the intensity with which the foot hits the floor, the amplitudes of the exciter signals are modulated by the peak value of the 1-norm of the acceleration vector measured between two subsequent activations of the calcaneous pressure sensor as illustrated in FIG. 4. The same signal may be used for both the auditory and tactile feedback in order to mimic the real-life scenario, where the same source of vibration produces acoustic and tactile cues.

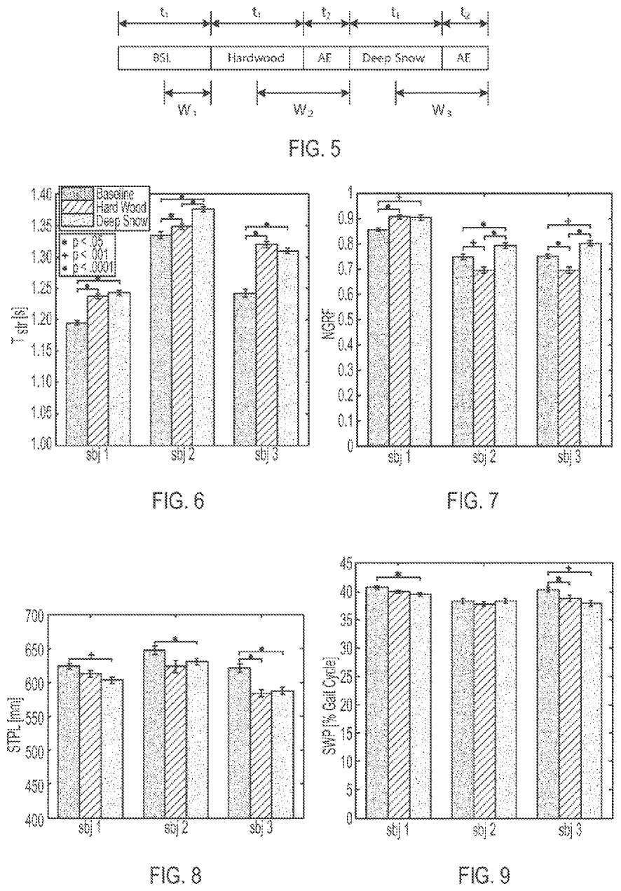

An experimental gait training and analysis system was tested to determine whether the rendering of different ground surface compliance through audio-tactile underfoot feedback may alter the natural gait pattern of a subject. A 6-cm long and 2.3-m wide rectangular circuit was traced on a floor. Subjects wearing the system were asked to walk approximately along the track in a counter-clockwise direction. Reflective markers were placed on the subject's feet and shanks to measure ankle plantar/dorsi-flexion angle and the kinematics of the feet. A rail-mounted motion capture system with eight cameras was used to track the markers at a sample rate of 100 Hz. The protocol included three 3-minute long sessions, as illustrated in FIG. 5, where t1 represents a time period of 180-seconds, t2 represents a time period of 90-seconds, and W1-W3 represents analyzed time windows. The first session (BSL) was a baseline session during which feedback was disabled. During a second session (Hard Wood), the feedback engine simulated walking on a hard surface. During a third session (Deep Snow), the feedback engine simulated walking on an aggregate material. After the second and third sessions, a 90-second session with no feedback was included to analyze potential after effects (AE) of the previous audio-tactile feedback.

Stride time (Tstr), normalized swing period (SWP) and normal ground reaction force (NGRF) at initial contact (IC) were estimated from the readings of the piezo-resistive sensors of the footwear units. Stride time is defined as the time elapsed between two subsequent peaks of the heel signal. Normalized swing period is defined as the peak value of the heel signal over the gait cycle. Step length (STPL) was compute as the projection of the horizontal displacement of a heel marker onto the plane of progression between initial contact of one leg and the subsequent initial contact of the contralateral leg.

In Deep Snow mode (i.e., aggregate material, soft simulated compliance), the audio-tactile feedback significantly decreased cadence with respect to the baseline gait, resulting in increased Tstr, as illustrated in FIG. 6. The magnitude of the normal ground reaction forces at initial contact, as estimated by NGRF, also increased as compared to baseline values, as illustrated in FIG. 7, while step length decreased significantly, as illustrated in FIG. 8. These changes were consistent across the three subjects tested, although two subjects also showed a significant reduction of normalized swing period, as shown in FIG. 9.

Results were more mixed for the simulated hard surface (Hard Wood). While Tstr significantly increased in all subjects, step length showed decreasing trends, but changes were significant for subject 3 only while the changes for the others were close to significance. Additionally, this mode significantly altered NGRF in all three subjects. While subjects 2 and 3 reduced impact force, an opposite effect was found in subject 1.

Step height and range of motion of ankle plantar-dorsi flexion were also investigated. Even though both variables showed a decreasing trend from Baseline to Hard Wood and from the latter to Deep Snow, none of these differences reached significance. Significant differences between the two feedback modalities were detected in NGRF. Both subjects 2 and 3 showed smaller impact forces when the rendering of the hard surface was active compared to when the rendering of the aggregate material was active.

Overall, these results suggest that ecological underfoot audio-tactile feedback may significantly alter the natural gait cycle of subjects. Between the two tested feedback modes, the feedback corresponding to aggregate material was more effective in impacting the subject's gait, especially with respect to variables STPL and SWP. In addition, the concurrent auditory and vibrotactile feedback may be more effective than auditory feedback alone in impacting the subject's gait. Results on impact forces at initial contact suggest that opposite effects may be evoked on the subject's gait when switching from the rendering of a hard surface to the rendering of a compliant one. Thus, a decrease in the peak ground reaction at initial contact may be induced by a simulated hard walking surface, and a corresponding increase may be induced by a simulated soft walking surface.

Referring to FIGS. 10A-10D, a system 400 for gait training and analysis is shown. Similar to the system 300 illustrated in FIGS. 3A-3D, the system 400 may include two footwear units 402a, 402b and a processing module 460 attached to the belt 370 of the subject. Each footwear unit 402a, 402b measures pressure under the foot and kinematic data of the foot. The data is sent wirelessly (e.g., via wireless connections 452) to a portable single-board computer 464 attached to the belt 370, where the audio-tactile feedback is generated in real-time and converted to analog signals by a sound card 462. Each footwear module 402a, 402b may also include a driver box secured to the lateral posterior side of each module and contains three, 2-channel audio amplifier boards 330 to power the transducers 324-328.

Audio cables 350 (e.g., stereo audio cables similar to those used in headphones) carry the analog signals from the processing module 460 to each footwear unit 402a, 402b, where they are amplified (e.g., by one or more amplifiers 330) and fed to vibrotactile transducers 324-328 embedded in the sole. Audio feedback may be provided via headphones (not shown). When headphones are not used, a miniature loudspeaker 336 optionally attaches to an anterior strap of the footwear unit 402a, 402b and may be directly powered from the driver box.

Piezo-resistive force sensors 314-317 are attached to or embedded in the sole of each footwear unit 402a, 402b. The signals are digitized and sent to processing module 464 via a microcontroller 444 (e.g., 32-bit ARM Cortex-M4 processor). This unit 444 was encased on a heel-mounted box, along with a 3-axis accelerometer 448 and a Wi-Fi antenna. A multi-degree-of-freedom (DOF) inertial measurement unit 440 (IMU), for example, a 9-DOF IMU, may be mounted in the sole along the midline of the foot, below the tarsometatarsal articulations. A second inertial unit 442 may be secured to the subject's proximal shank, for example, with leg strap 372, as illustrated in FIG. 10B. A base sensor 446, such as an ultrasonic sensor, may be mounted on the medial-posterior side of the sole to estimate the base of walking, as illustrated in FIG. 10D.

The single-board computer 464 that attaches to the subject's belt 370 may be powered by a battery 468 (e.g., a lithium ion polymer (LiPo) battery) that fits on the top of the computer's enclosure. The battery 468 may power both the processing unit 460 and the footwear units 402a, 402b, or each footwear module may be provided with their own independent battery 348. A real-time dataflow programming environment running in the computer 464 manages the audio-tactile footstep synthesis engine and also performs data-logging (e.g., at 500 Hz) of pressure data and kinematic data on a memory device, for example, a microSD card. Modification of the feedback parameters may be accomplished by sending string commands to the computer 464 wirelessly or via an optional wired input. The multi-channel sound card 462 of the processing module 460 may attach to the belt 370 together with the computer 464, as illustrated in FIG. 10B.

The gait analysis and training system 400 illustrated in FIGS. 10A-10D is capable of estimating temporal and spatial gait parameters. The use of force resistive sensors (FRS), such as piezo-resistive sensors, is known to accurately estimate temporal gait parameters. The accuracy and precision of spatial parameters was thus separately assessed. These spatial parameters include ankle plantar-dorsiflexion angle (including ankle range of motion, or range of motion (ROM), and ankle symmetry), foot trajectory (including stride length and foot-ground clearance) and step width.

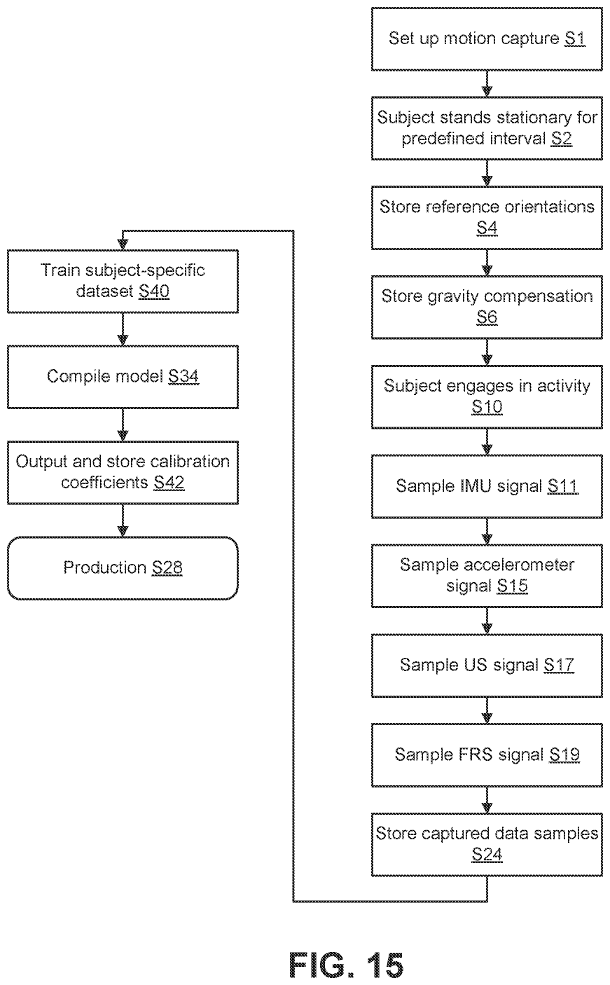

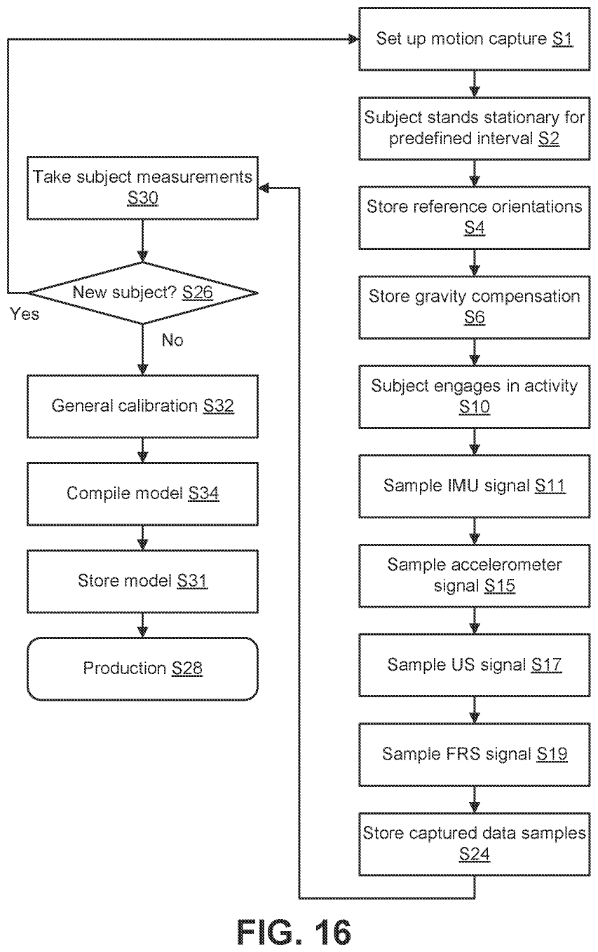

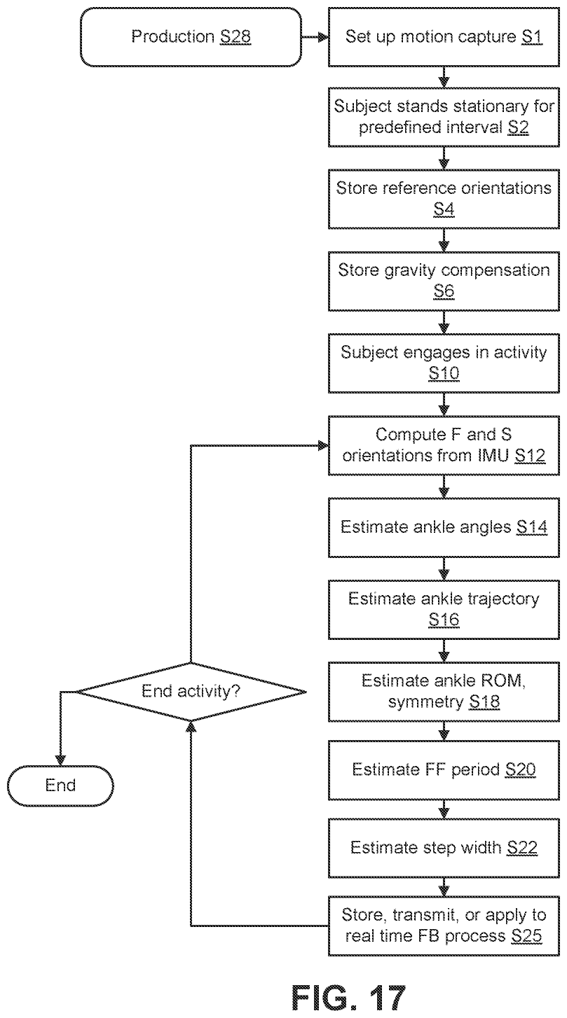

Each of the inertial measurement units (i.e., foot IMU 440 and shank IMU 442) provides orientation estimation relative to a reference (tare) frame based on an on-board EKF algorithm that weights the contributions of the accelerometer and magnetometer based on the current dynamics experienced by the inertial sensor within a subject-selectable range of feasible weights. The foot IMU 440 may be embedded in the footwear unit sole, with the local axis \hat{z}_{F} orthogonal to the sole and pointing downward and the local axis \hat{x}_{F} aligned with the longitudinal axis of the footwear unit. Referring to FIGS. 15 and 16, which relate to data capture, reduction, and calibration for subject-specific and generic training respectively, at startup, a subject stands stationary for a predefined interval such as 5-seconds S2 and the reference orientations for the foot and shank IMUSs are established and stored S4 in a memory or nonvolatile store (further detailed below). The mean acceleration values measured in the startup interval define the direction of the gravity vector g relative to the local IMU frames of foot and shank. Corresponding numerical compensation data may be stored at S6. The reference frame of the foot {F0} is defined as:

$${z}_{F 0} = g , {x}_{F 0} = \frac{{\hat{x}}_{F 0} - ( {\hat{x}}_{F 0} \cdot {z}_{F 0} ) {z}_{F 0}}{‖ {\hat{x}}_{F 0} - ( {\hat{x}}_{F 0} \cdot {z}_{F 0} ) {z}_{F 0} ‖} , {y}_{F 0} = {z}_{F 0} \times {x}_{F 0} , \qquad ( 1 )$$

where \hat{x}_{F0} is the local axis \hat{x}_{F} at t=0. The shank IMU is attached to the subject's proximal shank, for example, with a Velcro wrap. The local axis \hat{x}_{S} is assumed to be aligned with the longitudinal axis of the tibia, pointing upward, and the local axis \hat{z}_{S} is directed posteriorly. Similarly to the foot, the reference frame of the shank {S0} is defined as:

$${x}_{S 0} = - g , {z}_{S 0} = \frac{{\hat{z}}_{S 0} - ( {\hat{z}}_{S 0} \cdot {x}_{S 0} ) {x}_{S 0}}{‖ {\hat{z}}_{S 0} - ( {\hat{z}}_{S 0} \cdot {x}_{S 0} ) {x}_{S 0} ‖} , {y}_{S 0} = {z}_{S 0} \times {x}_{S 0} , \qquad ( 2 )$$

with \hat{z}_{S0} being the local axis \hat{z}_{S} at t=0. Assuming neutral subtalar position and neutral knee alignment during the taring process, the mapping between {F0} and {S0} is given by the following anti-diagonal matrix:

$${}_{F}^{S} {}_{0}^{0} R = [ \begin{matrix}0 & 0 & - 1 \\ 0 & - 1 & 0 \\ - 1 & 0 & 0\end{matrix} ] . \qquad ( 3 )$$

For t>0, the orientation estimations of foot and shank relative to their respective reference frames are returned in terms of yaw-pitch-roll Euler angles. The subject may begin walking activity at S10. The foot and shank orientations may be computer at S12. Together with (3), these data are sufficient to derive the three ankle angles: abduction/adduction, inversion/eversion and plantar/dorsiflexion which may be generated in real time by the on-board processor 460 at S14. The ankle plantar/dorsiflexion angle γ_{PD} may be most critical for gait propulsion and support against gravity, where γ_{PD} is defined as the relative pitch angle between foot and shank, offset by π/2. As shown by (3), the axes y_{S0} and y_{F0} are antiparallel, yielding

γ_{PD}=θ_{F}+θ_{S}, (4)

where θ_{F} and θ_{S} are the pitch angles of the foot and shank, respectively. For each leg, the ankle angle (4) is segmented into gait cycles (GC) using the readings of the heel pressure sensors as detectors of initial contact (IC). At S16, ankle trajectory is generated. For the i-th stride of each leg, the ankle angle is then time-normalized over the GC and downsampled into N equally spaced points to yield the ankle trajectory γ̅_{PDi}. At S18 ankle range of motion and symmetry are generated. The ankle range of motion ROMᵢ is defined as the difference between the absolute maximum and minimum of γ̅_{PDi}. A gait symmetry metric SYM, is derived as the RMS deviation between the normalized ankle trajectories of the right and left legs, corresponding to two consecutive strides:

$${SYM}_{i} = \sqrt{\frac{{\sum }_{j = 1}^{N} {( {\overline{γ}}_{PD \_ LEFTi , j} - {\overline{γ}}_{PD \_ RIGHTi , j} )}^{2}}{N}} , \qquad ( 5 )$$

with N being the number of samples in γ̅_{PDi}.

The foot IMU returns the components of the acceleration vector a (compensated by the gravity component) in the reference frame {F0}. A threshold-based algorithm detects the FF period as the fraction of the stance phase wherein the Euclidean norm of a is smaller than a predefined threshold. First, the foot velocity in the i-th stride vᵢ is obtained by integration of a, with the medians of the i-th and (i+1)-th FF periods defining the i-th interval of integration:

$${v}_{i , j} = {v}_{0 i} + \frac{1}{{f}_{s}} {\sum }_{k = {FF}_{i}}^{{FF}_{i} + j - 1} {a}_{k} , j ∈ [ 1 , {FF}_{i + 1} - {FF}_{i} + 1 ] , \qquad ( 6 )$$

v_{i,j} is the linear velocity of the foot in the j-th sample of the i-th stride, and [FFᵢ,FFᵢ₊₁] is the interval of integration for the i-th stride. The constant of integration v₀ᵢ is set to zero (ZUPT technique) and the raw velocity estimate (6) is corrected to compensate for velocity drift (assumed linear):

$${\overline{v}}_{i , j} = {v}_{i , j} - \frac{j - 1}{{FF}_{i + 1} - {FF}_{i}} {v}_{i , {FF}_{i + 1} - {FF}_{i} + 1} \qquad ( 7 )$$

The foot displacement dᵢ is computed by integration of v̅ᵢ:

$${d}_{i , j} = \frac{1}{{f}_{s}} {\sum }_{k = 1}^{j} {\overline{v}}_{i , j} , j ∈ [ 1 , {FF}_{i + 1} - {FF}_{i} + 1 ] , \qquad ( 8 )$$

where d_{i,j} is the displacement of the foot in the j-th sample of the i-th stride. dᵢ is known in {F0}, however, for the purposes of gait analysis, the reference frame {Di} aligned with the direction of progression is more desirable:

$${x}_{Di} = \frac{{d}_{i , {FF}_{i + 1} - {FF}_{i} + 1} - {d}_{i , 1}}{‖ {d}_{i , {FF}_{i + 1} - {FF}_{i} + 1} - {d}_{i , 1} ‖} , \\ {z}_{Di} = - \frac{{z}_{F 0} - ( {z}_{F 0} \cdot {x}_{Di} ) {x}_{Di}}{‖ {z}_{F 0} - ( {z}_{F 0} \cdot {x}_{Di} ) {x}_{Di} ‖} , {y}_{Di} = {z}_{Di} \times {x}_{Di} \qquad ( 9 )$$

d̅ᵢ—the sagittal-plane, normalized foot trajectory for the i-th stride—is obtained by projecting dᵢ onto the x_{Di}z_{Di} plane, time-normalizing over the interval [1,FFᵢ₊₁−fFᵢ+1], and downsampling into N equally-spaced points. Finally, stride length SLᵢ and foot ground clearance SHᵢ are defined as

$${SL}_{i} = \vert {\overline{d}}_{i , N} ( x ) - {\overline{d}}_{i , 1} ( x ) \vert , {SH}_{i} = {max}_{j ∈ [ 1 , N ]} ( {\overline{d}}_{i , j} ( z ) ) , \qquad ( 10 )$$

with d̅_{i,j}(x) and d̅_{i,j}(z) being the projections of d̅_{i,j} onto x_{Di} and z_{Di}, respectively.

Step width may be estimated S22 as the foot separation at mid-swing. During overground walking in a straight-line, the ultrasonic sensor mounted on the medial posterior site of the left sole returns a minimal distance when the forward swinging left foot passes the stance foot. The step width of the i-th stride SWᵢ is therefore estimated by the absolute minimum of the ultrasonic sensor readings during the swing phase of the i-th left stride.

The raw metrics described above may be affected by systematic and random errors. Not only may these errors be quantified experimentally by comparison with the data collected by a laboratory-grade motion capture system, but the same data may also be used to calibrate the less accurate wearable gate analsys system, largely compensating for the systematic errors and thereby improving the level of agreement between the two gait analysis systems. To this end, data were collected from fourteen healthy adult individuals with no gait abnormalities (10 males, 4 females, age 26.6±4.2 years, height 1.70±0.10 m, weight 64.9±9.5 kg, US shoe size 8.0±2.5).

Reflective markers were placed on both legs, either on anatomical landmarks at 502 (medial and lateral malleoli and femoral condyles, distal and proximal tibia) or on the footwear units at 504, 506 (close to the hallux, the calcaneus, and the heads of the 1st, 2nd and 5th metatarsal), as illustrated in FIG. 11. Prior to the test, subjects stood stationary for 5 seconds, at which time the on-board inertial sensors (e.g., IMU 440 and IMU 442) were zeroed at this time. Subjects completed 30 laps at a self-selected, comfortable pace. During each lap, subjects walked along a 14 m long, straight-line path marked on the floor, made a clockwise turn, and went back to the starting point. Each session lasted approximately 15 minutes. Subjects' movements were simultaneously recorded by wearable gait analysis system 400 and a separate camera-based motion capture system with 10 cameras. Sampling rates were set as 500 Hz for the gait analysis system 400 and as 100 Hz for the camera-based system. An infrared LED controlled by gait analysis system 400 was used to sync the two systems. A 5-m section in the middle of the first leg of each lap was regarded as representative of steady state walking, and the corresponding strides were included in the analysis described below.

Gait parameters estimated by gait analysis system 400 may be divided into scalar parameters (i.e., N=1 sample per stride) and vector parameters (i.e., N=101 samples per stride, uniformly distributed in the interval 0-100% GC). Stride length (SL), foot ground clearance (SH), base of walking (SW), ankle symmetry (SYM) and ankle range of motion (ROM) belong to the first group. Vector parameters include ankle angle (γ_{PD}) and foot trajectory (d̅=[d̅(x)d̅(z)]). The calibration approach described below applies to both groups. The raw metrics from the gait analysis system 400 and the data from the camera-based system were processed using custom MATLAB code. The training datasets pₜᵣ^{V} and pₜᵣ^{S} (where the superscripts V and S indicate the reference system and system 400, respectively) were obtained for each subject and each parameter by selecting every other stride from the full set of data, while the remaining data formed the testing datasets pₜₛ^{V} and pₜₛ^{S}. Prior to the actual calibration, an optimization script was implemented to determine the order and the cutoff frequency of the low-pass Butterworth filter (8 Hz, 4th order) applied to the norm of the foot acceleration ∥a∥, and the optimal threshold used to estimate FF periods from the measured acceleration. This optimization was based exclusively on training data. Then, two alternative calibration approaches were implemented as described in the following.

Subject-specific calibration includes the training dataset of a specific participant S40 and outputs a set of calibration coefficients S42 that are tailored to that subject. Data samples from IMUs S11, accelerometer S15, ultrasound sonar S17, and force resistive sensors S10 may be stored S24 and employed to create subject-specific calibrated models or generic models as described. In practice, this approach may be applied if a camera-based motion capture system is available to the experimenter, and calibration data may be easily collected from the subject prior to the use of gait analysis system 400. For each parameter p, N linear regression models were generated in the form of:

pₜᵣ^{V}(j)˜pₜᵣ^{S}(j), j∈[1,N], (11)

where pₜᵣ*(j) is the j-th sample of p measured by the gait analysis system 400 or by the camera-based reference system. These models yielded β_{0,j} and β_{1,j}, the optimal coefficients (in the least square sense) which minimize the sum of the squared residuals. The estimate of p at the i-th stride was computed as:

\hat{p}ᵢ^{S}(j)=β_{0,j}+β_{1,j}p_{ts,i}^{S}(j), j∈[1,N], (12)

and the associated error was calculated as:

eᵢ(j)=\hat{p}ᵢ^{S}(j)−p_{ts,i}^{V}(j), j∈[1,N] (13)

This approach was independently applied to each subject's dataset.

As for generic calibration (FIG. 16), for each subject, the calibration coefficients were computed based on the training datasets of all the other subjects, and the testing data of the excluded subject were used for validation (leave-one-out cross validation, or LOOCV). Subject athropmetric measurements are obtained for each subject and stored S30 and the characterstics used to compile a generic model S34 adjusted by anthropometric characteristics (see below) to process real-time data inputs during production runs. In practice, generic type of calibration is representative of the general application of gait analysis system 400, when it is impractical or unfeasible to perform a subject-specific calibration prior to using the system 400. In this case, the basic linear model was augmented with the subjects' anthropometric characteristics listed below:

pₜᵣ^{V}(j)˜pₜᵣ^{S}(j)+Height+Weight+Shoe Size+Age+Gender, j∈[1,N], (14)

Solving the least square problem yielded m+2 regression coefficients (β₀ . . . βₘ₊₁), with m=5 being the number of anthropometric characteristics included in the model. The estimate of p at the i-th stride was computed as:

$${\hat{p}}_{i}^{S} ( j ) = {β}_{0 , j} + {β}_{1 , j} {p}_{ts , i}^{S} ( j ) + {\sum }_{k = 1}^{5} {β}_{k + 1 , j} {x}_{k} , j ∈ [ 1 , N ] , \qquad ( 15 )$$

where xₖ is the covariate related to the k-th anthropometric characteristic. In validation experiments, this procedure was iterated 14 times, once for each subject. In a production system, the subjects contributing to the generic model would be a variegated population selected to form the generic model which is iterated through S26 to generate and store S31 a basis model for future subjects in production uses of the model by subjects not used in the calibration.

TABLE 1
Calibration results (mean RMSE ± SD)

| | Units | Symbol | Subject Specific | Generic |
|---|---|---|---|---|
| Ankle ROM | [deg] | ROM | 2.12 ± 0.63 | 4.76 ± 1.91 |
| Ankle Symmetry | [deg] | SYM | 1.95 ± 0.38 | 2.72 ± 1.53 |
| Stride length | [cm] | SL | 2.30 ± 0.90 | 2.93 ± 1.32 |
| Foot-ground Clearance | [cm] | SH | 0.38 ± 0.10 | 0.70 ± 0.37 |
| Base of Walking | [cm] | SW | 0.82 ± 0.19 | 1.54 ± 0.70 |
| Ankle Angle | [deg] | γ_{PD} | 2.70 ± 0.39 | 4.33 ± 1.01 |
| Foot Trajectory | [cm] | d̅ | 3.30 ± 0.32 | 4.53 ± 0.90 |

Note that other anthropometric characteristics may be used to augment the model such as hip circumference, waist circumference, whether and to what degree the subject has arthritis in the hip or knee joints, estimate of the symmetry of the arthritis. These characteristics can be defined as broad classes and may rely on variable judgment of the estimator, and they need not be precisely discriminated yet still enhance the model's accuracy in the estimation of gait kinematics.

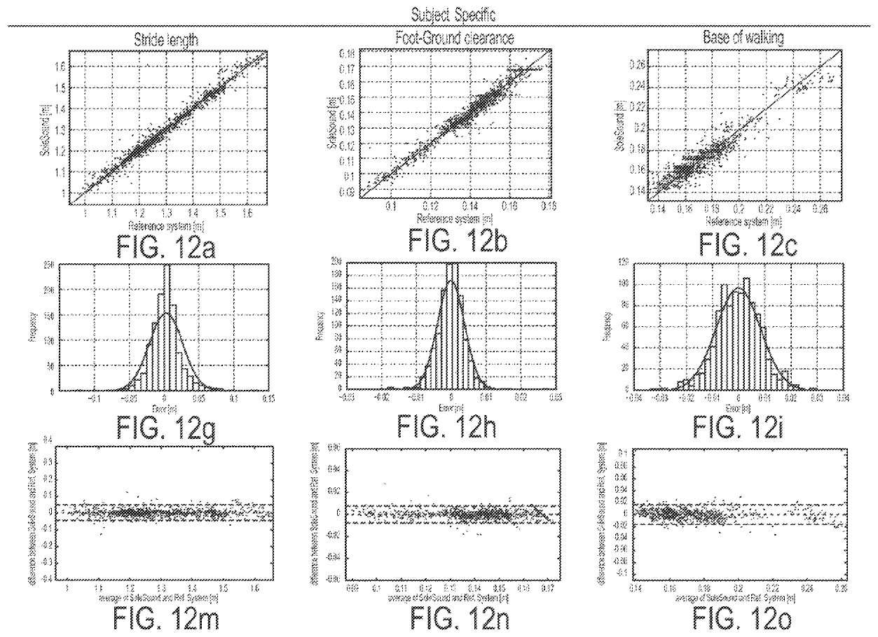

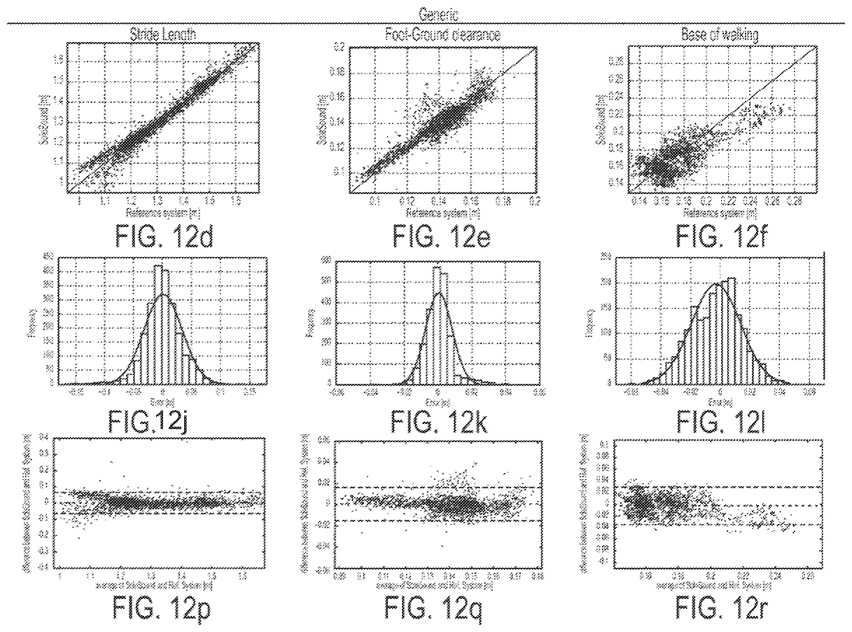

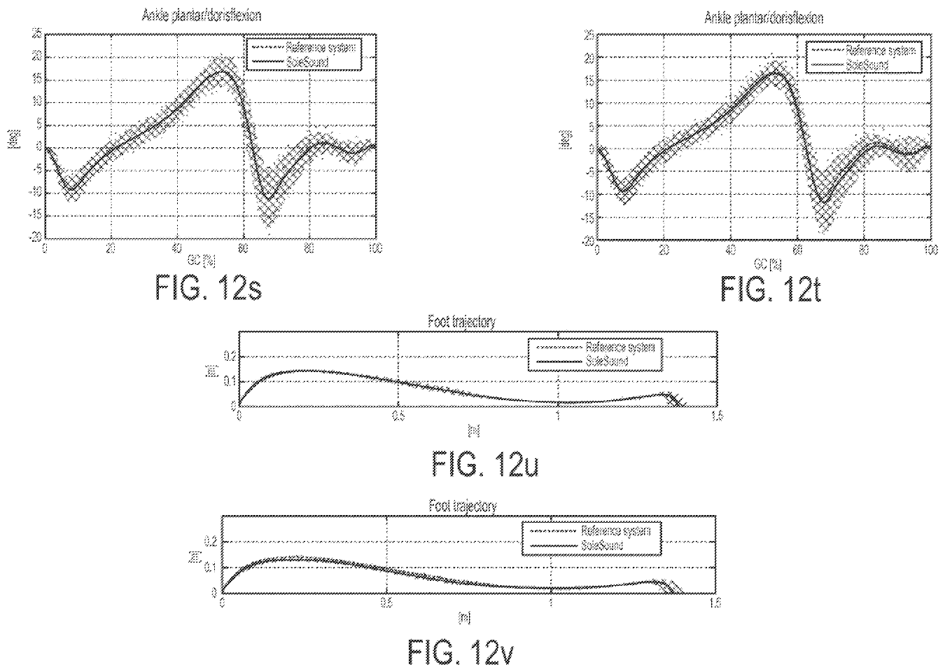

A total of 1888 strides was acquired by gait analysis system 400 and by the camera-based reference system (i.e., 4-5 gait cycles for each of the 30 laps, for each subject). Results are reported in Table 1 in terms of (mean RMSE±SD) for both calibration strategies. FIGS. 12a through 12v show the correlation plots between the gait analysis system 400 and the camera-based reference system (FIGS. 12a-12f), the frequency distribution of the measurement error (FIGS. 12g-12l) and the Bland-Altman plots (FIGS. 12m-12r) for a subset of the scalar parameters. FIGS. 12s-t show the ankle dorsiflexion angle averaged across all subjects, and FIGS. 12u-12v illustrate the average foot trajectory for a representative subject. Shaded areas indicate +/−1 SD. The performances of wearable devices may be reported in terms of accuracy and precision (mean error±SD) rather than in terms of RMSE. This alternative convention is directly related to the diagrams shown in FIGS. 12g-12l. Under this convention, the results reported in Table 1 translate as: 0.27±2.40 cm for SL, −0.01±0.39 cm for SH, −0.01±0.84 cm for SW in the case of the subject-specific calibration. The corresponding values for the generic calibration are: 0.01±3.28 cm for SL, 0.06±0.79 cm for SH, and −0.30±1.65 cm for SW.

According to embodiments of the disclosed subject matter, the gait analysis system may measure two types of gait parameters: spatial parameters, which include stride length, foot-ground clearance, base of walking, foot trajectory, and ankle plantar-dorsiflexion angle; and temporal parameters, which include cadence, single/double support, symmetry ratios, and walking speed. Wireless communication and data logging are performed at 500 Hz, a sampling rate which help reduce latency in the sound feedback.

Precise alignment of IMUs and anatomical segments usually requires preliminary calibration steps, which may be accomplished either with custom-made jigs or with a camera based motion capture system, by rigidly attaching a cluster of reflective markers to the mounting plate of each inertial sensor. These steps must be completed prior to each experimental session to guarantee the level of accuracy reported. Such methods reduce the portability of the wearable system. However, in the calibration method presented here, markers may be placed exclusively on anatomical landmarks, thus making the reported results independent of precise alignment of the IMUs to the human limbs.

Instead of relying on professional-grade inertial sensors to improve the system's performance, embodiments of the disclosed gait analysis system may achieve the same target using mid-grade, cost-effective IMUs, by adopting linear calibration techniques. After deriving linear models based on raw datasets and corresponding reference datasets (as discussed in above), linear corrections were successfully used to reduce systematic errors. Even though calculation of the linear models is carried out off-line, applying the models requires minimal computational cost, and is therefore suitable for real-time applications using micro-controllers.

The estimates of stride length, foot ground clearance and base of walking demonstrate a good level of agreement, as indicated by the Bland-Altman plots (FIGS. 12(m)-(r)). For the stride length, better results were obtained in terms of accuracy and precision compared to similar shoe-based systems. The RMSE on the estimation of the foot trajectory obtained with the gait analysis system are deemed acceptable, being smaller than 2.5% SL and 3.5% SL for the subject-specific calibration and the generic calibration, respectively. The capability of measuring the base of walking and spatiotemporal gait symmetry are additional novel aspects.

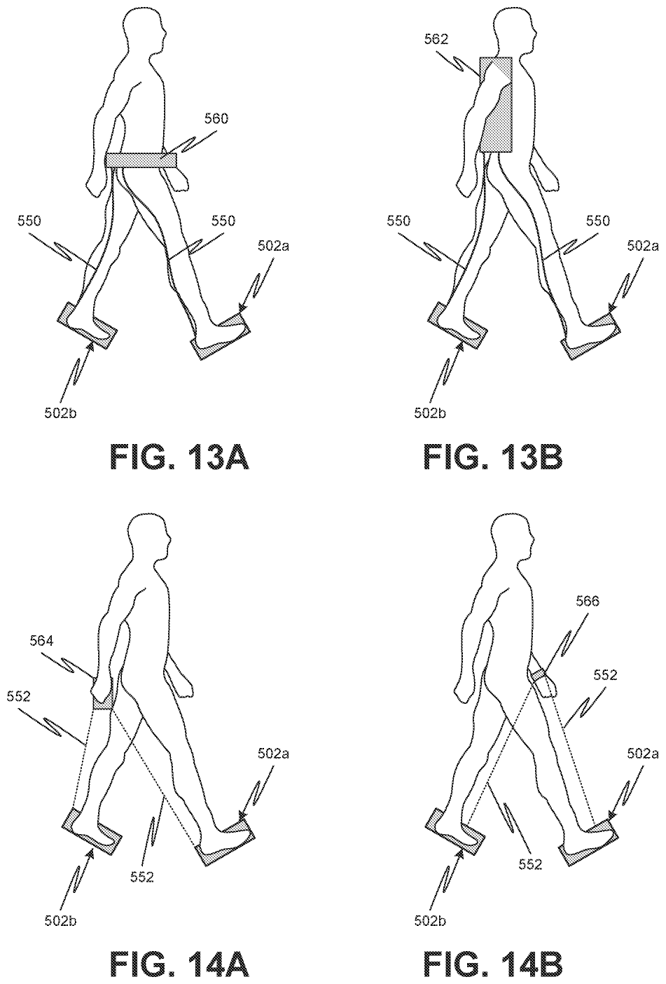

Referring to FIG. 13A, in one or more embodiments of the disclosed subject matter, a gait analysis system may have a pair of footwear modules 502a, 502b with sensing and feedback components worn by a subject and a belt-mounted processing module 560 that processes sensor signals and generates feedback signals. As noted above, sensor signals may be conveyed wirelessly from the footwear units 502a, 502b to the belt-mounted processing module 560, while audio cables 550 convey the feedback signals from the processing module 560 to the footwear units 502a, 502b. In an alternative configuration illustrated in FIG. 13B, the processing module 562 may be worn by the subject as a backpack rather than a belt-mounted unit.

Although a hybrid wired-wireless connection is discussed above for communication between the footwear units and the processing modules, it is also possible to have a completely wireless (or a completely wired) connection between the footwear unit and processing modules, according to one or more contemplated embodiments. In one or more contemplated embodiments, the processing module may be configured as a handheld device (e.g., a Smartphone 564) or a wearable component (e.g., wristwatch 566) that receives sensor signals from and communicates feedback signals to the footwear units 502a, 502b via a wireless connection (e.g., Bluetooth), as illustrated in FIGS. 14A-14B.

In one or more first embodiments, a gait training and analysis system may be worn by a subject and may comprise a pair of footwear modules, a processing module, and audio cables. Each footwear module may be constructed to be worn on a foot of the subject and may comprise a sole portion, a heel portion, a speaker, and a wireless communication module. The sole portion may have a plurality of piezo-resistive pressure sensors and a plurality of vibrotactile transducers. Each piezo-resistive sensor may be configured to generate a sensor signal responsively to pressure applied to the sole portion. Each vibrotactile transducer may be configured to generate vibration responsively to one or more feedback signals. The heel portion may have a multi-degree of freedom inertial sensor. The speaker may be configured to generate audible sound in response to the one or more feedback signals. The wireless communication module may be configured to wirelessly transmit each sensor signal. The processing module constructed to be worn as a belt by the subject. The processing module may be configured to process each sensor signal received from the wireless communication module and to generate the one or more feedback signals responsively thereto. The audio cables may connect each footwear module to the processing module and may be configured to convey the one or more feedback signals from the processing module to the vibrotactile transducers and speakers of the footwear unit.

In the first embodiments, or any other embodiment, for each footwear module, a respective one of the piezo-resistive sensors is located underneath the calcaneous, the head of the 4^{th} metatarsal, the head of the 1^{st} metatarsal, and the distal phalanx of the hallux of each foot.

In the first embodiments, or any other embodiment, for each footwear module, a first one of the vibrotacticle transducers is located underneath an anterior aspect of the calcaneous, a second one of the vibrotacticle transducers is located underneath a posterior aspect of the calcaneous, a third one of the vibrotacticle transducers is located underneath the middle of the lateral arch, a fourth one of the vibrotacticle transducers is located underneath the head of the 1^{st} metatarsal, and a fifth one of the vibrotacticle transducers is located underneath the distal phalanx of the hallux of each foot.

In the first embodiments, or any other embodiment, for each footwear module, a first of the feedback signals drives the first and second vibrotactile transducers, a second of the feedback signals drives the third the vibrotactile transducers, a third of the feedback signals drives the fourth and fifth vibrotactile transducers, and a fourth of the feedback signals drives the speaker.

In the first embodiments, or any other embodiment, the inertial sensor is a nine-degree of freedom inertial sensor.

In the first embodiments, or any other embodiment, for each footwear module, the inertial sensor is located along the midline of the foot below the tarsometatarsal articulations.

In the first embodiments, or any other embodiment, the processing module is configured to determine one or more gait parameters responsively to the sensor signals. The gait parameters comprise stride length, foot-ground clearance, base of walking, foot trajectory, ankle plantar-dorsiflexion angle, cadence, single/double support, symmetry ratios, and walking speed.

In the first embodiments, or any other embodiment, the processing module comprises on-board memory for storing the determined gait parameters.

In the first embodiments, or any other embodiment, the processing module includes a single-board computer and a sound card.

In the first embodiments, or any other embodiment, the system further comprises ultrasonic sensors. Each ultrasonic sensor may be coupled to the sole portion of a respective one of the footwear units. Each ultrasonic sensor may be configured to detect a base which the sole of the respective footwear module contacts during walking.

In the first embodiments, or any other embodiment, the system further comprises a second inertial sensor coupled to a proximal shank of the subject.

In the first embodiments, or any other embodiment, the system further comprises accelerometers. Each accelerometer may be coupled to the heel portion of a respective one of the footwear units.

In the first embodiments, or any other embodiment, the processing module is configured to sample data at a rate of at least 500 Hz.

In the first embodiments, or any other embodiment, each footwear module comprises a power source and the processing module comprises a separate power source.

In the first embodiments, or any other embodiment, each power source is a lithium ion polymer battery.

In the first embodiments, or any other embodiment, the processing module is configured to change the one or more feedback signals responsively to gait pattern changes or intensity of impact so as to produce different sounds or vibrations from each footwear module.

In one or more second embodiments, a system for synthesizing continuous audio-tactile feedback in real-time may comprise one or more sensors and a computer processor. The one or more sensors are configured to be attached to footwear of a subject to measure pressure under the foot and/or kinematic data of the foot. The computer processor is configured to be attached to the subject to receive data from the one or more sensors and to generate audio-tactile signals based on the received sensor data. The generated audio-tactile signal is transmitted to one or more vibrotactile transducers and loudspeakers included in the footwear unit.

In the second embodiments, or any other embodiment, the computer processor is configured to be attached to a belt of the subject.

In the second embodiments, or any other embodiment, the one or more sensors include piezo-resistive force sensors.

In the second embodiments, or any other embodiment, the computer processor is a single-board computer processor.

In one or more third embodiments, a method for real-time synthesis of continuous audio-tactile feedback comprises measuring pressure and/or kinematic data of a foot of a subject, and sending the pressure and/or kinematic data to a computer processor attached to a body part of the subject to generate audio-tactile feedback signal based on the measured pressure and/or kinematic data. The method may further comprise sending the audio-tactile feedback signal to vibrotactile sensors attached to the foot of the subject.

In the third embodiments, or any other embodiment, the sending the pressure and/or kinematic data is performed wirelessly.

In the third embodiments, or any other embodiment, the sending the audio-tactile feedback signal is via audio cables.

In one or more fourth embodiments, a system comprises one or more footwear modules and a wearable processing module. Each footwear module comprises one or more pressure sensors, one or more inertial sensors, and feedback module. The feedback module is configured to provide a wearer of the footwear unit with at least one of auditory and tactile feedback. The wearable processing module is configured to receive signals from the pressure and inertial sensors and to provide one or more command signals to the feedback module to generate the at least one of auditory and tactile feedback responsively to the received sensor signals.

In the fourth embodiments, or any other embodiment, the one or more pressure sensors is at least four pressure sensors.

In the fourth embodiments, or any other embodiment, a first of the pressure sensors is located underneath the calcaneous, a second of the pressure sensors is located underneath the head of the 4th metatarsal, a third of the pressure sensors is located underneath the head of the 1st metatarsal, and a fourth of the pressure sensors is located underneath the distal phalanx of the hallux of a foot of the wearer.

In the fourth embodiments, or any other embodiment, the one or more pressure sensors comprise one or more piezo-resistive force sensors.

In the fourth embodiments, or any other embodiment, the one or more inertial sensors is a nine-degree of freedom inertial measurement unit.

In the fourth embodiments, or any other embodiment, one of the inertial sensors is located at a midline of a foot of the wearer below the tarsometatarsal articulations.

In the fourth embodiments, or any other embodiment, the system further comprises a second inertial sensor mounted on the wearer remote from the one or more footwear modules.

In the fourth embodiments, or any other embodiment, the second inertial sensor is coupled to a proximal shank of the wearer.

In the fourth embodiments, or any other embodiment, the one or more footwear modules comprise a base sensor configured to detect a surface on which a bottom of the footwear unit contacts during walking.

In the fourth embodiments, or any other embodiment, the base sensor is an ultrasonic sensor.

In the fourth embodiments, or any other embodiment, the one or more footwear modules include an accelerometer.

In the fourth embodiments, or any other embodiment, the accelerometer is disposed proximal to the heel of the one of more footwear modules.

In the fourth embodiments, or any other embodiment, the one or more footwear modules comprises a plurality of vibration transducers.

In the fourth embodiments, or any other embodiment, a first one of the vibration transducers is located underneath an anterior aspect of the calcaneous, a second one of the vibration transducers is located underneath a posterior aspect of the calcaneous, a third one of the vibration transducers is located underneath the middle of the lateral arch, a fourth one of the vibration transducers is located underneath the head of the 1st metatarsal, and a fifth one of the vibration transducers is located underneath the distal phalanx of the hallux of each foot.

In the fourth embodiments, or any other embodiment, the feedback module comprises a speaker.

In the fourth embodiments, or any other embodiment, a first of the command signals drives the first and second vibration transducer, a second of the command signals drives the third vibration transducer, a third of the command signals drives the fourth and fifth transducers, and a fourth of the command signals drives the speaker.

In the fourth embodiments, or any other embodiment, the plurality of vibration transducers is at least five transducers for each footwear module.

In the fourth embodiments, or any other embodiment, the vibration transducers are arranged anteriorly, posteriorly, and under the lateral arch of a foot of the wearer.

In the fourth embodiments, or any other embodiment, the anteriorly arranged vibration transducers are driven by a first of the command signals, the posteriorly arranged vibration transducers are driven by a second of the command signals, and the vibration transducers under the lateral arch are driven by a third of the command signals.

In the fourth embodiments, or any other embodiment, the feedback module comprises a speaker.

In the fourth embodiments, or any other embodiment, the one or more footwear modules are configured to transmit sensor signals to the wearable processing module via a wireless connection.

In the fourth embodiments, or any other embodiment, the system further comprises one or more audio cables coupling the wearable processing module to the one or more footwear modules, wherein the one or more command signals are transmitted via the one or more audio cables.

In the fourth embodiments, or any other embodiment, the wearable processing module is constructed to be worn as or attached to a belt or a backpack of the subject.

In the fourth embodiments, or any other embodiment, the wearable processing module is configured to wirelessly communicate with an external network or computer.

In the fourth embodiments, or any other embodiment, the wearable processing module is configured to determine at least one gait parameter and to generate data responsively to the sensor signals.

In the fourth embodiments, or any other embodiment, the wearable processing module comprises memory for storing the generated data.

In the fourth embodiments, or any other embodiment, the gait parameters include one or more of spatial and temporal parameters.

In the fourth embodiments, or any other embodiment, the spatial parameters include stride length, foot-ground clearance, base of walking, foot trajectory, and ankle plantar-dorsiflexion angle.

In the fourth embodiments, or any other embodiment, the temporal parameters include cadence, single/double support, symmetry ratios, and walking speed.

In the fourth embodiments, or any other embodiment, the wearable processing module is configured to sample data at a rate of at least 500 Hz.

In the fourth embodiments, or any other embodiment, each of the footwear unit and processing modules has a separate power supply.

In the fourth embodiments, or any other embodiment, each power supply is a lithium-ion polymer battery.

In the fourth embodiments, or any other embodiment, the processing module comprises a multi-channel sound card that generates analog command signals.

In the fourth embodiments, or any other embodiment, the one or more footwear modules comprises a sole with the one or more pressure sensors embedded therein.

In the fourth embodiments, or any other embodiment, the one or more command signals change responsively to gait pattern changes or intensity of impact of the one or more footwear modules so as to produce different sounds and/or vibrations via the feedback module.

In the fourth embodiments, or any other embodiment, the feedback module is located on a perimeter of a foot inserted into the respective footwear module.

In one or more fifth embodiments, a method for gait analysis and/or training comprises generating auditory feedback via one or more speakers and/or tactile feedback via one or more vibrotactile transducers of the footwear unit. The generating is responsive to signals from pressure and inertial sensors of the footwear unit indicative of one or more gait parameters.

In the fifth embodiments, or any other embodiment, the method further comprises wirelessly transmitting the sensor signals from the footwear unit worn by a subject to a remote processor worn by the subject.

In the fifth embodiments, or any other embodiment, the method further comprises transmitting via one or more wired connections signals from the remote processor to the footwear unit that generate the auditory and/or tactile feedback.

In the fifth embodiments, or any other embodiment, the method further comprises determining one or more gait parameters selected from stride length, foot-ground clearance, base of walking, foot trajectory, ankle plantar-dorsiflexion angle, cadence, single/double support, symmetry ratios, and walking speed.

In the fifth embodiments, or any other embodiment, the method further comprises storing the determined gait parameters as data in memory of the remote processor.

In the fifth embodiments, or any other embodiment, the method further comprises wirelessly transmitting the stored data to a separate computer or network.

In the fifth embodiments, or any other embodiment, the method further comprises attaching a first footwear module to a right foot of a subject and a second footwear module to a left foot of the subject, attaching a remote processor to a belt worn by the subject, and coupling audio cables between the remote processor and the first and second footwear modules.

In the fifth embodiments, or any other embodiment, the coupling audio cables comprises positioning audio cables along respective legs of the subject.

In the fifth embodiments, or any other embodiment, the method further comprises positioning an inertial measurement unit along a leg of the subject.

In the fifth embodiments, or any other embodiment, the generating is further responsive to signals from the inertial measurement unit.

In the fifth embodiments, or any other embodiment, the generating auditory feedback is via one or more speakers of the footwear unit.

In the fifth embodiments, or any other embodiment, the generating auditory feedback is via headphones worn by the subject.

According to sixth embodiments, the disclosed subject matter includes a method (or a system adapted) for providing feedback for support of gait training. The method or system includes or is adapted for capturing gait kinematics of a subject with a reference system. Simultaneously with the capturing, inertial signals are sampled that indicate orientation and displacement motion of a gait of a subject from a N-degree of freedom inertial measurement unit (IMU) mounted in the middle of the sole of each of two sensor footwear unit worn by the subject and an IMU worn on each shank of the subject. Also simultaneously with the capturing, the sonar signals are also sampled, the sonar signals indicating a separation between legs using at least one ultrasonic range sensor (SONAR) on at least one of the two footwear unit. Also simultaneously with the capturing, force signals are sampled from force sensors (FRS) located at multiple points on soles of the two sensor footwear unit. Anthropometric characteristics of the subject are stored on a computer and a model is generated to estimate gait characteristics from the captured gait kinematics, the anthropometric characteristics of the set of subjects, and the samples resulting from all of the sampling. The model is stored on a wearable processor worn by the subject. Instrumented footwear units configured as the sensor footwear units worn by the subject during the actions (a) through (e) are attached to the subject and the wearable processor is connected to the instrumented footwear units. Using the wearable processor, kinematics of gait of the subject are estimated responsively to the model and sonar, inertial, and force signals from the instrumented footwear unit worn by the subject and an IMU worn on the subject's shank. Feedback signals may be generated responsively to signals resulting from at least one of the SONAR, FRS, and IMU sensors and/or the kinematics of gait and outputting the feedback signals to a user interface worn by the subject.

Further sixth embodiment may be modified to form additional sixth embodiments in which the user interface includes headphones and the feedback signals include audio signals representing characteristics of a walkable surface selected and stored in the wearable processor. Further sixth embodiment may be modified to form additional sixth embodiments in which the user interface includes speakers in one or both of the instrumented footwear units and the feedback signals includes audio signals representing characteristics of a walkable surface selected and stored in the wearable processor. Further sixth embodiment may be modified to form additional sixth embodiments in which the user interface includes one or more vibrotactile transducers in the instrumented footwear units and the feedback signals includes haptic feedback representing characteristics of a walkable surface selected and stored in the wearable processor.

Further sixth embodiment may be modified to form additional sixth embodiments in which the reference system includes a video-based motion capture system. Further sixth embodiment may be modified to form additional sixth embodiments in which the gait kinematics includes data indicating stance width. Further sixth embodiment may be modified to form additional sixth embodiments in which anthropometric characteristics include subject height. Further sixth embodiment may be modified to form additional sixth embodiments in which anthropometric characteristics include subject weight. Further sixth embodiment may be modified to form additional sixth embodiments in which gait characteristics include stride length. Further sixth embodiment may be modified to form additional sixth embodiments in which the gait characteristics include foot trajectory. Further sixth embodiment may be modified to form additional sixth embodiments in which the gait characteristics include ankle range of motion. Further sixth embodiment may be modified to form additional sixth embodiments in which the gait characteristics include ankle plantar/dorsiflection range of motion and instantaneous ankle angle relative to a reference direction. Further sixth embodiment may be modified to form additional sixth embodiments in which feedback signals include tactile feedback or audible sound delivered through transducers in the sensor footwear unit. Further sixth embodiment may be modified to form additional sixth embodiments in which wearable processor is in a wearable unit.

Further sixth embodiment may be modified to form additional sixth embodiments in which the model is a linear model. Further sixth embodiment may be modified to form additional sixth embodiments in which IMU has 9 degrees of freedom responsive to derivatives of rotational and translational displacement and magnetic field orientation. Further sixth embodiment may be modified to form additional sixth embodiments in which the estimating includes detecting events by thresholding respective ones of the signals. Further sixth embodiment may be modified to form additional sixth embodiments in which thresholding includes discriminating an interval of a gait cycle during which the feet of the subject are flat on the floor. Further sixth embodiment may be modified to form additional sixth embodiments in which the capturing gait kinematics of a subject with a reference system includes indicating transient positions of anatomical features. Further sixth embodiment may be modified to form additional sixth embodiments in which the anatomical features are generated from markers located directly on the anatomical features of the subject. Further sixth embodiment may be modified to form additional sixth embodiments in which the capturing gait kinematics and the estimating kinematics of gait each include estimating one or more of ankle range of motion, ankle symmetry, stride length, foot-ground clearance, base of walking, ankle trajectory, and foot traj ectory.

Further sixth embodiment may be modified to form additional sixth embodiments in which at least one of the vibrotactile transducers and/or speakers connected to the footwear unit are integrated in the footwear unit. Further sixth embodiment may be modified to form additional sixth embodiments in which both the vibrotactile transducers and/or speakers are vibrotactile transducers and speakers connected to the footwear unit. Further sixth embodiment may be modified to form additional sixth embodiments in which both the vibrotactile transducers and/or speakers are vibrotactile transducers and speakers connected to the footwear unit integrated in the footwear unit. Further sixth embodiment may be modified to form additional sixth embodiments in which the vibrotactile transducers and/or speakers are connected to a wearable sound synthesizer by a cable. Further sixth embodiment may be modified to form additional sixth embodiments in which the anthropometric characteristics include at least one of subject height, weight, shoe size, age, and gender. Further sixth embodiment may be modified to form additional sixth embodiments in which anthropometric characteristics include subject height, weight, shoe size, age, and gender. Further sixth embodiment may be modified to form additional sixth embodiments in which anthropometric characteristics include at least one of subject height, weight, hip circumference, shank length, thigh length, leg length, shoe size, age, and gender. Further sixth embodiment may be modified to form additional sixth embodiments in which estimating kinematics of gait and generating feedback signals are performed with a wearable system on battery power that is not tethered to a power source or separate computer. Further sixth embodiment may be modified to form additional sixth embodiments in which anthropometric characteristics include at least one of subject dimensions, weight, gender, and/or pathology and estimate of a degree of the pathology.

Further sixth embodiment may be modified to form additional sixth embodiments in which SONAR indicates the separation between the feet. Further sixth embodiment may be modified to form additional sixth embodiments in which there are SONAR sensors on each footwear unit and the measure of the leg separation is indicated by processing signals from the SONAR sensors by taking the minimum physical separation between the near-most obstacle detected by each SONAR sensor as an indication of the leg separate. Further sixth embodiment may be modified to form additional sixth embodiments in which the kinematics of gait of the new subject include stride length. Further sixth embodiment may be modified to form additional sixth embodiments in which the kinematics of gait of the new subject foot trajectory. Further sixth embodiment may be modified to form additional sixth embodiments in which the kinematics of gait of the new subject ankle range of motion. Further sixth embodiment may be modified to form additional sixth embodiments in which the kinematics of gait of the new subject include ankle plantar/dorsiflection range of motion and instantaneous ankle angle relative to a reference direction. Further sixth embodiment may be modified to form additional sixth embodiments in which the generating feedback signals includes generating sounds responsive to a selectable command identifying a surface type and responsive to instantaneous signals from the FRSs. Further sixth embodiment may be modified to form additional sixth embodiments in which the footwear unit further includes a further inertial sensor. Further sixth embodiment may be modified to form additional sixth embodiments in which the footwear unit includes at least 3 FRS sensors. Further sixth embodiment may be modified to form additional sixth embodiments in which the footwear unit includes at least 5 FRS sensors. Further sixth embodiment may be modified to form additional sixth embodiments in which the footwear unit includes multiple vibrotactile transducers located at multiple respective positions in the sole of the footwear unit.

According to seventh embodiments, the disclosed subject matter includes a method for providing feedback for support of gait training. Gait kinematics of a subject are captured with a reference system. Simultaneously with the capturing, inertial signals are sampled indicating orientation and displacement motion of a gait of a subject from a N-degree of freedom inertial measurement unit (IMU) mounted in the middle of the sole of each of two sensor footwear unit worn by the subject and an IMU worn on each shank of the subject. Simultaneously with the capturing, sonar signals are sampled which indicate a separation between legs using at least one ultrasonic range sensor (SONAR) on at least one of the two footwear unit. Simultaneously with the capturing, force signals are sample from force sensors (FRS) located at multiple points on soles of the two sensor footwear unit. Anthropometric characteristics of the subject are stored on a computer after measuring them. These steps are repeated for each member of a set of subjects with varied anthropometric characteristics and a model is generated to estimate gait characteristics from the captured gait kinematics, the measured anthropometric characteristics of the set of subjects, and the samples resulting from all of the sampling obtained for all the subjects in the set whereby the model predicts parameters representing gait characteristics responsively to both samples from sensor signals and the anthropometric characteristics of a new subject. The new subject's anthropometric characteristics are measured, where the new subject is outside the set used to generate the model. The new subject is fitted with instrumented footwear units configured as the sensor footwear unit and worn by the subjects in the set. Using a wearable processor connected to the instrumented footwear units, the kinematics of gait of the new subject are estimated responsively to the model and anthropometric characteristics of the new subject, and sonar, inertial, and force signals from instrumented footwear units worn by the new subject and an IMU worn on the new subject's shank. This may be done by a wearable computer or on a separate host processor or server. Feedback signals may be generated of the responsively to signals resulting from at least one of the SONAR, FRS, and IMU sensors and/or the kinematics of gait or the signals may be stored or transmitted to a separate server or host for processing. Both of these can also be done in further embodiments.

Further seventh embodiment may be modified to form additional seventh embodiments in which the one or storing and generating feedback signals responsively to signals resulting from at least one of the SONAR, FRS, and IMU sensors and/or the kinematics of gait includes generating feedback signals responsively to signals resulting from at least one of the SONAR, FRS, and IMU sensors and/or the kinematics of gait and the user interface includes headphones and the feedback signals include audio signals representing characteristics of a walkable surface selected and stored in the wearable processor. Further seventh embodiment may be modified to form additional seventh embodiments in which the one or storing and generating feedback signals responsively to signals resulting from at least one of the SONAR, FRS, and IMU sensors and/or the kinematics of gait includes generating feedback signals responsively to signals resulting from at least one of the SONAR, FRS, and IMU sensors and/or the kinematics of gait and the user interface includes headphones and the feedback signals includes audio signals representing characteristics of a walkable surface selected and stored in the wearable processor.

Further seventh embodiment may be modified to form additional seventh embodiments in which the one or storing and generating feedback signals responsively to signals resulting from at least one of the SONAR, FRS, and IMU sensors and/or the kinematics of gait includes generating feedback signals responsively to signals resulting from at least one of the SONAR, FRS, and IMU sensors and/or the kinematics of gait and the user interface includes headphones and the feedback signals includes haptic feedback representing characteristics of a walkable surface selected and stored in the wearable processor. Further seventh embodiment may be modified to form additional seventh embodiments in which the reference system includes a video-based motion capture system. Further seventh embodiment may be modified to form additional seventh embodiments in which the gait kinematics includes data indicating stance width. Further seventh embodiment may be modified to form additional seventh embodiments in which the anthropometric characteristics include subject height. Further seventh embodiment may be modified to form additional seventh embodiments in which the anthropometric characteristics include subject weight. Further seventh embodiment may be modified to form additional seventh embodiments in which the gait characteristics include stride length. Further seventh embodiment may be modified to form additional seventh embodiments in which the gait characteristics include foot trajectory. Further seventh embodiment may be modified to form additional seventh embodiments in which the gait characteristics include ankle range of motion. Further seventh embodiment may be modified to form additional seventh embodiments in which the gait characteristics include ankle plantar/dorsiflection range of motion and instantaneous ankle angle relative to a reference direction.

Further seventh embodiment may be modified to form additional seventh embodiments in which the feedback signals include tactile feedback or audible sound delivered through transducers in the sensor footwear unit. Further seventh embodiment may be modified to form additional seventh embodiments in which the wearable processor is in a wearable unit. Further seventh embodiment may be modified to form additional seventh embodiments in which the model is a linear model. Further seventh embodiment may be modified to form additional seventh embodiments in which the IMU has 9 degrees of freedom responsive to derivatives of rotational and translational displacement and magnetic field orientation. Further seventh embodiment may be modified to form additional seventh embodiments in which the estimating includes detecting events by thresholding respective ones of the signals. Further seventh embodiment may be modified to form additional seventh embodiments in which the thresholding includes discriminating an interval of a gait cycle during which the feet of the subject are flat on the floor. Further seventh embodiment may be modified to form additional seventh embodiments in which the capturing gait kinematics of a subject with a reference system includes indicating transient positions of anatomical features. Further seventh embodiment may be modified to form additional seventh embodiments in which the anatomical features are generated from markers located directly on the anatomical features of the subject.

Further seventh embodiment may be modified to form additional seventh embodiments in which the capturing gait kinematics and the estimating kinematics of gait each include estimating one or more of ankle range of motion, ankle symmetry, stride length, foot-ground clearance, base of walking, ankle trajectory, and foot trajectory. Further seventh embodiment may be modified to form additional seventh embodiments in which the one or storing and generating feedback signals responsively to signals resulting from at least one of the SONAR, FRS, and IMU sensors and/or the kinematics of gait includes generating feedback signals responsively to signals resulting from at least one of the SONAR, FRS, and IMU sensors and/or the kinematics of gait and the user interface includes headphones and wherein at least one of the vibrotactile transducers and/or speakers connected to the footwear unit are integrated in the footwear unit. Further seventh embodiment may be modified to form additional seventh embodiments in which the one or storing and generating feedback signals responsively to signals resulting from at least one of the SONAR, FRS, and IMU sensors and/or the kinematics of gait includes generating feedback signals responsively to signals resulting from at least one of the SONAR, FRS, and IMU sensors and/or the kinematics of gait and the user interface includes headphones and wherein both the vibrotactile transducers and/or speakers are vibrotactile transducers and speakers connected to the footwear unit.

Further seventh embodiment may be modified to form additional seventh embodiments in which the one or storing and generating feedback signals responsively to signals resulting from at least one of the SONAR, FRS, and IMU sensors and/or the kinematics of gait includes generating feedback signals responsively to signals resulting from at least one of the SONAR, FRS, and IMU sensors and/or the kinematics of gait and the user interface includes headphones and wherein both the vibrotactile transducers and/or speakers are vibrotactile transducers and speakers connected to the footwear unit integrated in the footwear unit. Further seventh embodiment may be modified to form additional seventh embodiments in which the one or storing and generating feedback signals responsively to signals resulting from at least one of the SONAR, FRS, and IMU sensors and/or the kinematics of gait includes generating feedback signals responsively to signals resulting from at least one of the SONAR, FRS, and IMU sensors and/or the kinematics of gait and the user interface includes headphones and wherein the vibrotactile transducers and/or speakers are connected to a wearable sound synthesizer by a cable.

Further seventh embodiment may be modified to form additional seventh embodiments in which the anthropometric characteristics include at least one of subject height, weight, shoe size, age, and gender. Further seventh embodiment may be modified to form additional seventh embodiments in which the anthropometric characteristics include subject height, weight, shoe size, age, and gender. Further seventh embodiment may be modified to form additional seventh embodiments in which the anthropometric characteristics include at least one of subject height, weight, hip circumference, shank length, thigh length, leg length, shoe size, age, and gender. Further seventh embodiment may be modified to form additional seventh embodiments in which the one or storing and generating feedback signals responsively to signals resulting from at least one of the SONAR, FRS, and IMU sensors and/or the kinematics of gait includes generating feedback signals responsively to signals resulting from at least one of the SONAR, FRS, and IMU sensors and/or the kinematics of gait and the user interface includes headphones and wherein the estimating kinematics of gait and generating feedback signals are performed with a wearable system on battery power that is not tethered to a power source or separate computer.

Further seventh embodiment may be modified to form additional seventh embodiments in which the anthropometric characteristics include at least one of subject dimensions, weight, gender, and/or pathology and estimate of a degree of the pathology. Further seventh embodiment may be modified to form additional seventh embodiments in which the SONAR indicates the separation between the feet. Further seventh embodiment may be modified to form additional seventh embodiments in which there are SONAR sensors on each footwear unit and the measure of the leg separation is indicated by processing signals from the SONAR sensors by taking the minimum physical separation between the near-most obstacle detected by each SONAR sensor as an indication of the leg separate. Further seventh embodiment may be modified to form additional seventh embodiments in which the kinematics of gait of the new subject include stride length. Further seventh embodiment may be modified to form additional seventh embodiments in which the kinematics of gait of the new subject foot trajectory.

Further seventh embodiment may be modified to form additional seventh embodiments in which the kinematics of gait of the new subject ankle range of motion. Further seventh embodiment may be modified to form additional seventh embodiments in which the kinematics of gait of the new subject include ankle plantar/dorsiflection range of motion and instantaneous ankle angle relative to a reference direction. Further seventh embodiment may be modified to form additional seventh embodiments in which the one or storing and generating feedback signals responsively to signals resulting from at least one of the SONAR, FRS, and IMU sensors and/or the kinematics of gait includes generating feedback signals responsively to signals resulting from at least one of the SONAR, FRS, and IMU sensors and/or the kinematics of gait and the user interface includes headphones and wherein the generating feedback signals includes generating sounds responsive to a selectable command identifying a surface type and responsive to instantaneous signals from the FRSs. Further seventh embodiment may be modified to form additional seventh embodiments in which the footwear unit further includes a further inertial sensor. Further seventh embodiment may be modified to form additional seventh embodiments in which the footwear unit includes at least 3 FRS sensors. Further seventh embodiment may be modified to form additional seventh embodiments in which the footwear unit includes at least 5 FRS sensors. Further seventh embodiment may be modified to form additional seventh embodiments in which the one or storing and generating feedback signals responsively to signals resulting from at least one of the SONAR, FRS, and IMU sensors and/or the kinematics of gait includes generating feedback signals responsively to signals resulting from at least one of the SONAR, FRS, and IMU sensors and/or the kinematics of gait and the user interface includes headphones and wherein the footwear unit includes multiple vibrotactile transducers located at multiple respective positions in the sole of the footwear unit.

According to eight embodiments, the disclosed subject matter includes a method for providing feedback for support of gait training. Gait kinematics of a subject are captured with a reference system. Simultaneously with the capturing, inertial signals are sampled indicating orientation and displacement motion of a gait of a subject from a N-degree of freedom inertial measurement unit (IMU) mounted in the middle of the sole of each of two sensor footwear unit worn by the subject and an IMU worn on each shank of the subject. Simultaneously with the capturing, sonar signals are sampled which indicate a separation between legs using at least one ultrasonic range sensor (SONAR) on at least one of the two footwear unit. Simultaneously with the capturing, force signals are sample from force sensors (FRS) located at multiple points on soles of the two sensor footwear unit. Anthropometric characteristics of the subject are stored on a computer. A model is generated to estimate gait characteristics from the captured gait kinematics, the anthropometric characteristics of the set of subjects, and the samples resulting from all of the sampling. Over a period of time, sensor data is sampled and stored which is responsive to sonar, inertial, and force signals of the subject instrumented footwear device described with respect to the calibration process. Time-dependent kinematic parameters are estimated representing the gait of the subject over the course of the period of time responsively to the model and the sensor data that has been stored. Thus, the system and method are like a holter monitor used for observing the heart of a patient. A wearable device can record all the readings, or reduced versions thereof, during the course of a period of time such as a day. The data recorded by the monitor can be stored and transmitted from the home of a subject, for example, to a computer accessible by a clinician who may process the data to provide time-based kinematic data for analysis of the subject.

Further eighth embodiment may be modified to form additional eighth embodiments in which the reference system includes a video-based motion capture system. Further eighth embodiment may be modified to form additional eighth embodiments in which the gait kinematics includes data indicating stance width. Further eighth embodiment may be modified to form additional eighth embodiments in which the gait characteristics include stride length. Further eighth embodiment may be modified to form additional eighth embodiments in which the gait characteristics include foot trajectory.

Further eighth embodiment may be modified to form additional eighth embodiments in which the gait characteristics include ankle range of motion. Further eighth embodiment may be modified to form additional eighth embodiments in which the gait characteristics include ankle plantar/dorsiflection range of motion and instantaneous ankle angle relative to a reference direction. Further eighth embodiment may be modified to form additional eighth embodiments in which the feedback signals include tactile feedback or audible sound delivered through transducers in the sensor footwear unit. Further eighth embodiment may be modified to form additional eighth embodiments in which the model is a linear model. Further eighth embodiment may be modified to form additional eighth embodiments in which the IMU has 9 degrees of freedom responsive to derivatives of rotational and translational displacement and magnetic field orientation. Further eighth embodiment may be modified to form additional eighth embodiments in which the estimating includes detecting events by thresholding respective ones of the signals.

Further eighth embodiment may be modified to form additional eighth embodiments in which the thresholding includes discriminating an interval of a gait cycle during which the feet of the subject are flat on the floor. Further eighth embodiment may be modified to form additional eighth embodiments in which the capturing gait kinematics of a subject with a reference system includes indicating transient positions of anatomical features.

Further eighth embodiment may be modified to form additional eighth embodiments in which the anatomical features are generated from markers located directly on the anatomical features of the subject. Further eighth embodiment may be modified to form additional eighth embodiments in which the capturing gait kinematics and the estimating kinematics of gait each include estimating one or more of ankle range of motion, ankle symmetry, stride length, foot-ground clearance, base of walking, ankle trajectory, and foot traj ectory.

Further eighth embodiment may be modified to form additional eighth embodiments in which the estimating kinematics of gait and generating feedback signals are performed with a wearable system on battery power that is not tethered to a power source or separate computer. Further eighth embodiment may be modified to form additional eighth embodiments in which the SONAR indicates the separation between the feet. Further eighth embodiment may be modified to form additional eighth embodiments in which there are SONAR sensors on each footwear unit and the measure of the leg separation is indicated by processing signals from the SONAR sensors by taking the minimum physical separation between the near-most obstacle detected by each SONAR sensor as an indication of the leg separate. Further eighth embodiment may be modified to form additional eighth embodiments in which the kinematics of gait of the subject include stride length.

Further eighth embodiment may be modified to form additional eighth embodiments in which the kinematics of gait of the subject foot trajectory. Further eighth embodiment may be modified to form additional eighth embodiments in which the kinematics of gait of the subject ankle range of motion. Further eighth embodiment may be modified to form additional eighth embodiments in which the kinematics of gait of the subject include ankle plantar/dorsiflection range of motion and instantaneous ankle angle relative to a reference direction. Further eighth embodiment may be modified to form additional eighth embodiments in which the generating feedback signals includes generating sounds responsive to a selectable command identifying a surface type and responsive to instantaneous signals from the FRSs. Further eighth embodiment may be modified to form additional eighth embodiments in which the footwear unit further includes a further inertial sensor. Further eighth embodiment may be modified to form additional eighth embodiments in which the footwear unit includes at least 3 FRS sensors. Further eighth embodiment may be modified to form additional eighth embodiments in which the footwear unit includes at least 5 FRS sensors.

In this application, unless specifically stated otherwise, the use of the singular includes the plural and the use of “or” means “and/or.” Furthermore, use of the terms “including” or “having,” as well as other forms, such as “includes,” “included,” “has,” or “had” is not limiting. Any range described herein will be understood to include the endpoints and all values between the endpoints.

Furthermore, the foregoing descriptions apply, in some cases, to examples generated in a laboratory, but these examples may be extended to production techniques. For example, where quantities and techniques apply to the laboratory examples, they should not be understood as limiting. In addition, although specific materials have been disclosed herein, other materials may also be employed according to one or more contemplated embodiments.

Features of the disclosed embodiments may be combined, rearranged, omitted, etc., within the scope of the invention to produce additional embodiments. Furthermore, certain features may sometimes be used to advantage without a corresponding use of other features.

It is thus apparent that there is provided in accordance with the present disclosure, system, methods, and devices for gait analysis and/or training. Many alternatives, modifications, and variations are enabled by the present disclosure. While specific embodiments have been shown and described in detail to illustrate the application of the principles of the present invention, it will be understood that the invention may be embodied otherwise without departing from such principles. Accordingly, Applicant intends to embrace all such alternatives, modifications, equivalents, and variations that are within the spirit and scope of the present invention.

The present section describes a Recurrent Neural Network classifier model that segments walking data recorded with instrumented footwear. The instrumented footwear is much like the Sole Sound instrumented footwear described above. The signals from three piezoresistive sensors, a 3-axis accelerometer, and Euler angles are used to generate temporal gait characteristics of a user. A greater or smaller number of sensors may be used in additional embodiments. The model was tested using a dataset collected from 28 healthy adults containing 4,198 steps. Errors were calculated with respect to an instrumented walkway. The mean error for heel strikes and toe offs were −5.9±37.1 ms and 11.4±47.4 ms respectively. These small errors show that the algorithm can be reliably used to segment the gait recordings and to use this segmentation to estimate temporal parameters of the participants. All sensor data from the instrumented footwear may be merged without preprocessing, or any human intervention, to generate gait characteristics. This greatly reduces the processing time and makes the technology amenable for real-time applications.
Recurrent Neural Networks and instrumented footwear can be combined together to generate reliable gait characteristics in near real-time.
DeepSole (a handy name for the footwear system) is a portable footwear system for gait characterization and is designed to be unobtrusive to the user and can be used outside of a clinic setting.
A Neural Network model can be used to segment gait information without needing specific calibration.

Above this section, the specification discloses systems, devices, and methods that may be adapted for use with the technology disclosed hereinbelow.

Gait analysis allows clinicians and researchers to quantitatively characterize the kinematics and kinetics of human movement. Sensor based gait characterization systems are recognized as clinical tools to analyze patient mobility. For example, quantitative gait data has been used to determine the need for surgery in children with Cerebral Palsy (CP) and to prescribe the care and treatment after surgery. Furthermore, it has been shown that children with CP who underwent clinical gait analysis before lower extremity orthopedic surgery had significantly lower incidence of additional surgery.

Devices that quantify gait can be either portable, such as instrumented shoes, or non-portable, such as motion capture systems and instrumented walkways. There is a tradeoff between these two classes of systems in terms of portability and accuracy. However, recent computer advances allow for the collection of meaningful data outside of the clinical setting, over different terrains and activities. This is critical for recording abnormal walking behaviors, e.g., episodic phenomena like freezing of gait of patients with Parkinson's Disease. Although the portable devices permit longer recordings in natural environments, the added flexibility increases the potential for sensor misinterpretation. This error can be significant when used on participants with irregular walking, such as the elderly, or individuals with CP, adding to the complexity of data processing.

Gait characterization typically includes both spatial and temporal parameters. These parameters can quantify changes in the user locomotion and can track progress of training or rehabilitation. For example, stride to stride fluctuations can be used to assess risk of falls and gait variability has been used as a good predictor for dementia.

To analyze the collected data, most techniques involve two stages: (i) segmenting the data into steps or strides to calculate temporal parameters, then (ii) estimating the spatial parameters using the segmented data. The initial contact time, usually made by the heel, is set as the start of the gait cycle. Different algorithms have been proposed to obtain gait characteristics, from simple thresholding algorithms to machine learning algorithms. These methods analyze the sensor readings but require human effort to validate and “clean” the data, e.g. for removing sensor errors or noise. This is a time intensive step and prone to errors as only a limited number of features during the sensor measurements can be considered, e.g., pressure or inertial measurements. The methods mentioned above provide good performance but rely on the skills of a person analyzing the data to find the important features in the recorded gait. Also, algorithms need to be formulated to identify these engineered features. The difficulty of finding these features increases as the number of sensors grows. However, limiting the number and types of sensors introduces the risk that data cannot be processed if the device malfunctions.

A model specifically created to reliably identify and characterize a person's gait using the raw data, without any pre-processing, greatly reduces the time needed to obtain meaningful data. This allows researchers and clinicians to record and analyze long walking sessions outside the clinical environment. However, it is useful for the model to maintain equivalent accuracy and precision when compared to the state-of-the-art methods, while still significantly reducing the processing time.

Machine learning allows the automation of tedious and time-consuming processes and greatly reduces the time needed to obtain meaningful output data. Convolution Neural Networks have been demonstrated to obtain spatiotemporal gait parameters from an inertial sensor with performance comparable to state-of-the-art devices. A gait segmentation algorithm using Hidden Markov Models (HMM) with signals acquired from a gyroscope mounted at the foot has been demonstrated with an accuracy of 98.3% when considering an event identified by a rejection window less than ±30 ms. But only three healthy participants were used walking on a treadmill for two minutes at various speeds and inclines.

Bayesian models have been used to estimate the temporal gait parameters of ten healthy participants over three 7.6 m laps at a comfortable walking speed. Only the acceleration data was recorded and processed, showing an accuracy and precision (absolute error±standard deviation) of 9.1±6.5 ms for step time, 42.3±20.2 ms for stance phase time, and 32.2±13.9 ms for swing time.

Artificial Neural Networks (ANN) allow the mapping of an input vector X to an output vector Y, where the input and output can be multidimensional. The algorithm looks at a single event through different sensors and merges this information in their mapping, thus avoiding the need to manually program algorithms that recognize engineered features. For time-series data, the ANN commonly used are either Convolutional Neural Networks (CNN) or Recurrent Neural Networks (RNN). CNN are specialized for processing data that have a grid-like topology and have been successfully used to identify human motion from the signal of several Inertial Measurement Units (IMU). RNN are models with the ability to sequentially process information one element at a time, generating a sequence-to-sequence mapping. They excel at determining outputs from inputs that are not independent. RNN are more desirable than CNN because they accumulate data, capturing long-range time dependencies.

The present disclosure presents an RNN model that classifies the recordings from an instrumented shoe. The model output is used to segment the walking data and to calculate temporal characteristics of the gait. RNN was chosen over CNN because it provides an output for every intermediate step of the network. This model property was used to reduce the number of incorrect predictions. The input to the network is the data of three pressure sensors, a 3-axis accelerometer, and Euler angles of the feet. Here, it is shown that using the RNN classifier, it is possible to segment the walking data within seconds without human intervention.

The dataset used for the training and evaluation of the model consists of 28 healthy participants over 18 years old (8 females and 20 males, age 19 to 31). A second dataset of 7 children (4 females and 3 males, age 7 to 14) with CP was collected and used for evaluation. Participant characteristics are listed in Table 2. Since the experiment of walking with shoes is non-invasive, the only requirement to participate in the experiment was the ability to walk independently for 6 minutes. None of the participants used assistive devices during their testing.

For the CP group, the inclusion criteria was that they were diagnosed with unilateral CP, were able to walk for 6 min without any assistance, cooperative, and aged between 6 and 17 years old. People that presented other neorulogical disorders, e.g., orthopedic surgery or botulinum toxin injections on the affected leg within 6 months were excluded from the experiment.

TABLE 2
Participant Characteristics for CP Group

| | Height | Weight | | | | Affected | | | Lesion |
|---|---|---|---|---|---|---|---|---|---|
| ID | (cm) | (kg) | Shoe | Gender | Age | Side | MACS | GMFCS | Type |
| CP001 | 185 | 94 | 12 | M | 15 | Left | II | I | MCA |
| CP002 | 170 | 52 | 12 | M | 14 | Left | II | I | PVL |
| CP003 | 132 | 24 | 6 | W | 10 | Left | II | II | PVL |
| CP004 | 152 | 52 | 6 | W | 12 | Right | I | I | MCA |
| CP005 | 137 | 42 | 5 | W | 8 | Left | III | II | PVL |
| CP006 | 138 | 27 | 5 | W | 9 | Left | III | II | PVL |
| CP007 | 155 | 33 | 7 | M | 14 | Left | II | I | PVL |

DeepSole is a new iteration of a modular instrumented footwear described herein. The earlier version was called SoleSound. DeepSole has several improvements in order to make it more portable, reliable, and durable. The system consists of two foot modules, each with a pressure sensitive insole, three vibration motors, a 9 DoF Inertia Measurement Unit (IMU) and a microcontroller, FIG. 1. The microcontrollers sample the sensors at 200 Hz, record the data to a MicroSD card and stream it over UDP for real-time visualization.

Each insole consists of three pressure areas: one located under the phalanges, second located under the metatarsals, and the third located under the calcaneus. The pressure sensors are made with a layer of piezoresistive fabric (Eontex, Calif.) in between two layer of conductive copper fabric. These sensors can be custom made to any shape and retain their piezoresistive properties. They provide an average loading of each independent area instead of just a single point. This feature is especially useful when characterizing populations with irregular loading during gait, such as children with CP. The vibration motors are located under the first and fifth metatarsals, and the calcaneus. Each can be controlled independently to change the vibration intensity. The system can be donned in minutes and is similar to putting on a regular pair of shoes. Due to the soft materials used, the insoles are indistinguishable by the wearer.

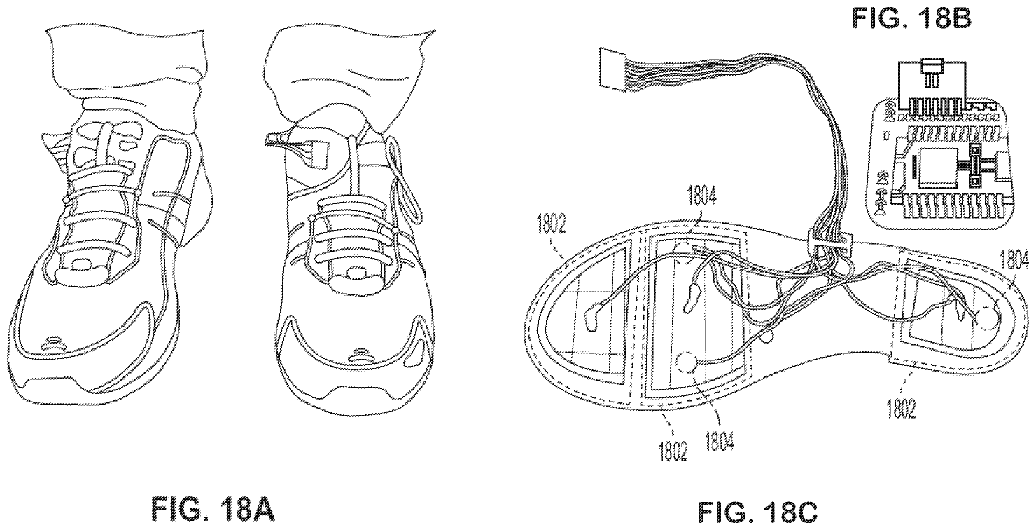

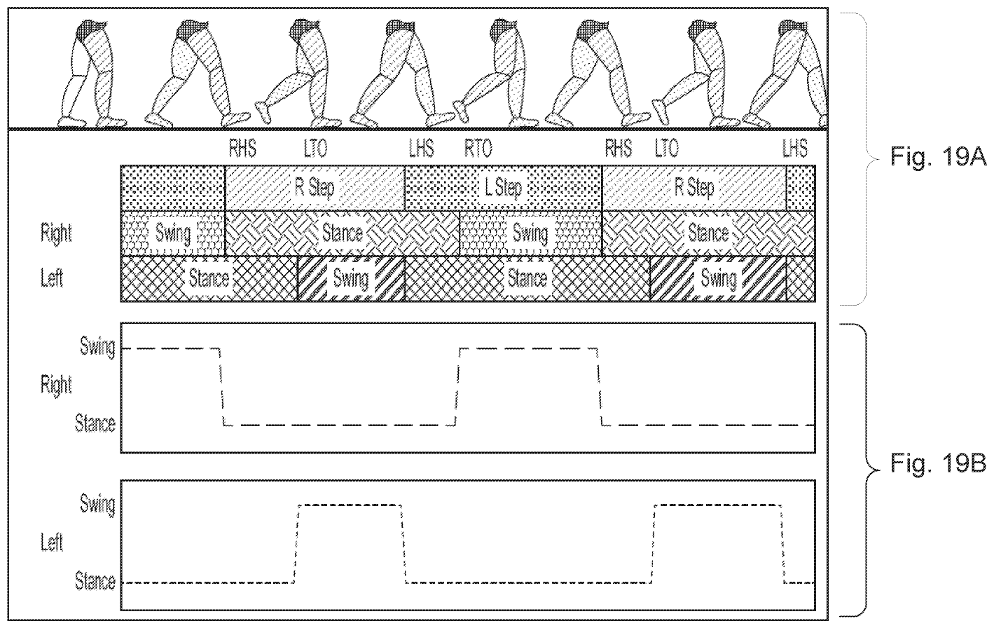

The participants were asked to perform the 6-minute walk test (6 MWT) while wearing the DeepSole system. During this test, a subject walked at a self-selected speed for 6 minutes in a hallway equipped with a Zeno Walkway (Protokinetics, PA). The walkway has a total length of 6 m, but 2 m were added to the extremes of the walkway to make a total walking distance of 10 m. Data was recorded simultaneously from both systems. FIG. 18A shows a subject wearing the DeepSole system. FIG. 18B shows a printed circuit (PCB) with microcontroller and IMU. FIG. 18C shows an instrumented insole with pressure sensors 1802 and vibration motors 1804.

Segmentation is the step of gait analysis that involves splitting the data into cycles. Each cycle is defined by Heel Strikes (HS) and Toe Offs (TO). Even though several algorithms exist to identify these events, they usually involve supervision and intervention from a human to identify faulty cycles. False positives can come either from sensor errors, or from gait variability of the participants. Identifying faulty cycles is time intensive and could take the user between 1 hour to 12+ hours to analyze 6 minutes of walking data of each subject.

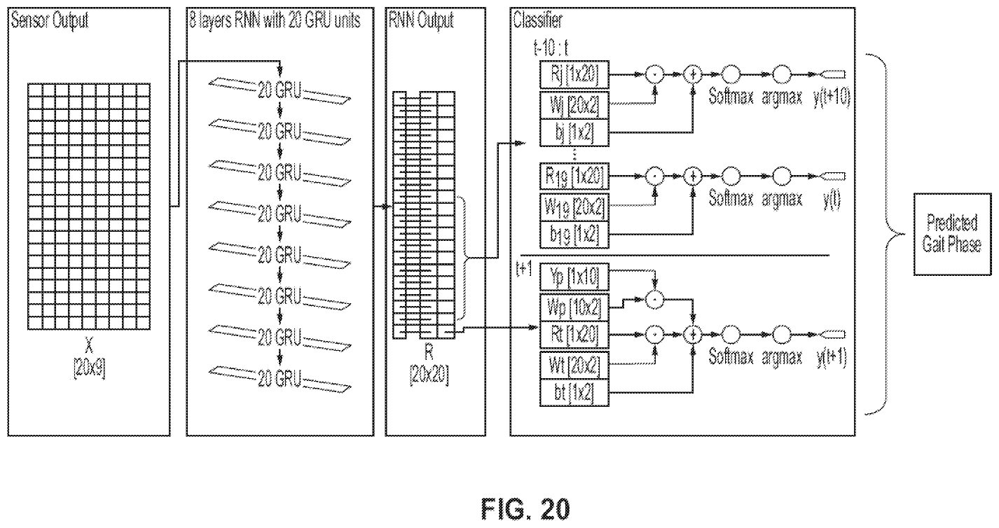

Using HS and TO, it is possible to segment data and calculate 15+ spatial gait parameters. A graphical example of the different gait events and how to identify these using only HS and TO events is shown in FIG. 19A shows a graphical representation of a normal gait cycle and how the events are defined by heel strikes and toe off. FIG. 19B shows an example of a binary function of the gait phases. The algorithm substitutes commonly used thresholding algorithm to segment the data. The thresholding algorithms are ineffective when the user has an abnormal gait, as the pressure data can be erratic and a single threshold value may not be sufficient for the entire recording.

The recordings were resampled from 200 Hz to 100 Hz to reduce the high frequency noise and the computational load. After this down-sampling, no other pre-processing was done except for appending the readings to create a matrix.

From the DeepSole, nine signals are obtained: three pressure sensor readings, three linear accelerations, and three Euler angles. The last 20 readings from the sensors are appended into a matrix X∈ [symbol] ^{20×9} to use as inputs to the RNN. Here, the columns represent the values of the signals and the rows represent the time when the signals were recorded. The last row is the current reading at time (t) and first row is the readings at time (t−19*dₜ), where dₜ is the sampling time of 10 ms. In the training set, the left and right side recordings were used indiscriminately. This allowed the model to classify the data using information only from the desired side. This makes the model suitable for predicting symmetric and asymmetric gait, as each side is predicted independently.

FIG. 20 illustrates a network architecture for the segmentation model. Sensor measurements are fed to a RNN with gated recurrent units (GRU), the output is then passed through a classifier to obtain the prediction for t+1.

Since HS and TO are very short time events, creating a model to identify these events would be impractical. Therefore, the gait cycle was split into the phases of a step and the HS and TO information were later reconstructed from this output. Using this approach, several training samples are obtained from a single step instead of only 2 per step, one for HS and one for TO. The Network is an RNN classifier with two classes: stance phase and swing phase. Using this strategy, the model can generate a function of time showing the phase of the gait. By using the differentiation of the output, HS is indicated as going from off the ground to on the ground (\dot{y}=−1), and TO as the point where the foot is no longer in contact with the ground (\dot{y}=1).

The output of the network is a binary function of time that shows the phases of the gait:

$$y ( t ) = \{ \begin{matrix}0 & Stance Phase \\ 1 & Swing Phase\end{matrix} \qquad ( 1 )$$

FIG. 20 shows a schematic of the model's architecture. First, the input matrix is normalized per channel and is fed into a RNN containing 8 layers, each with 20 GRU cells.
From the RNN, a matrix R∈ [symbol] ^{20×20} is obtained where every row i corresponds to the predicted value of y(i+1), and i=20 is equivalent to the current time t. This matrix is used in the classification layers.
At this point, the model splits into two outputs, one part gives the expected values for y(t) to (t−10) using rows i=9 to i=19 from matrix R and the following equations:

j=19−n (2)

y(t−n)=argmax (softmax(RⱼWⱼ+bⱼ)) (3)

where y(t−n) is the predicted value at time t−n, Rⱼ is the j^{th} row of matrix R, Wⱼ∈ [symbol] ^{20×2} is a weight matrix and bⱼ∈ [symbol] ^{1×2} is a bias vector.
The second output predicts the value of y(t+1) by considering the previous values of y using:

$$y ( t + 1 ) = arg max ( soft max ( {R}_{t} {W}_{t} + {y}_{p} {W}_{p} + {b}_{t} ) ) \qquad ( 4 )$$

$${y}_{p} = \{ \begin{matrix}{y}_{true} & if training \\ {y}_{predicted} & if evaluation\end{matrix} \qquad ( 5 )$$

where y(t+1) is the predicted time for the next location of the foot given the past 20 sensor readings, Rₜ is the last row of matrix R, Wₜ∈ [symbol] ^{20×2}, Wₚ∈ [symbol] ^{10×2} are weight matrices and bₜ∈ [symbol] ^{1×2} is a bias vector. yₚ is a row vector containing the last 10 values of the output y(t). During training, these values are fed from the training set, but during run and evaluation the predictions obtained from Eq. (3) are used.

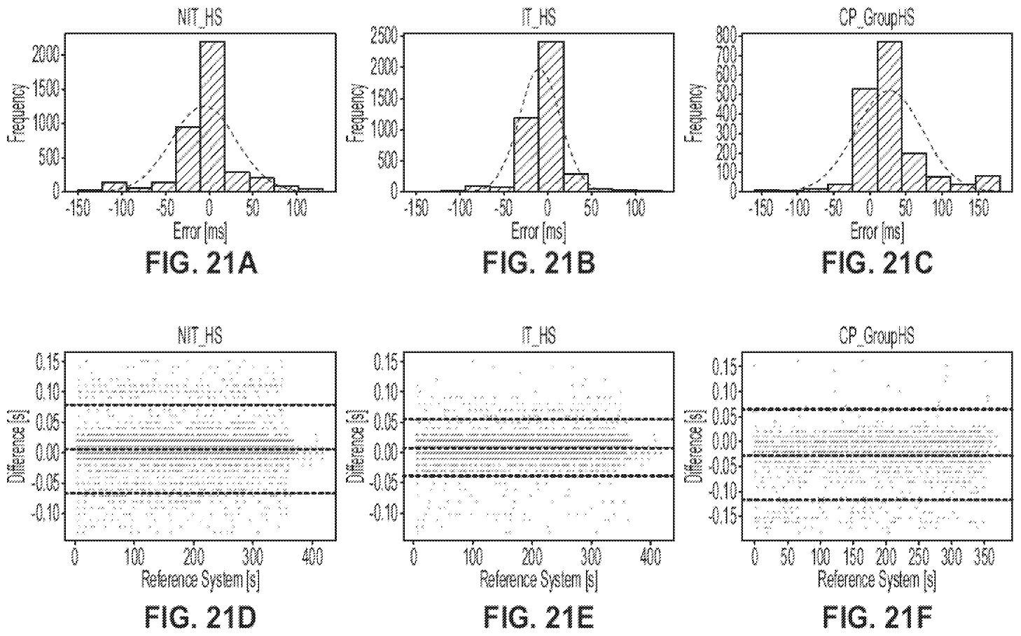

FIGS. 21A through 21C show error distributions of the identification errors for HS with respect to the reference system. FIGS. 21A, 21B, and 21C show a histogram of the error distributions for the three groups. The Bland-Altman plots showing the bounding error for HS for the NIT, IT, and CP groups are shown in 21D, 21E, and 21F, respectively. FIG. 21A shows is a NIT HS frequency histogram. FIG. 21B shows an IT HS frequency histogram. FIG. 21C shows a CP HS frequency histogram.

In equations (3), (4) the softmax activation and the argmax combined create a “1-of-2” encoding, winner-takes-all of the outputs. The softmax function is used to represent the probability distribution over two classes and argmax is used to choose the class with the highest probability.

Each model was trained over 200 epochs, i.e. the model goes 200 times through the dataset using an Adams optimizer to minimize the cross-entropy loss function (6).

$${H}_{{y}^{′}} ( y ) = - {\sum }_{i} {y}_{i}^{′} log ( {y}_{i} )$$

Google TensorFlow library was used to implement and train the network.

The model presented is a classifier of the gait phase, i.e., 0 for stance and 1 for swing. To obtain meaningful gait characteristics, one must identify the HS and the TO events.

Given the model architecture, at every time t, two outputs are provided, the predicted phase and the expected phase for the last 10 measurements. This means that after 10 system cycles, at every time t, there are 10 values for the position of the foot at time t. By rounding the mean of all ten predictions, the output can reduce the number of false predictions. This is particularly useful at the HS and TO gait events, since these are located at the transition between states and should be singleton events per step cycle.

To test the performance of the algorithm, a “leave-one-out cross-validation” (LOOCV) test was performed over the P participants (P=28). A total of P models were trained with P−1 participants. The LOOCV was repeated P times excluding a different subject for every iteration.

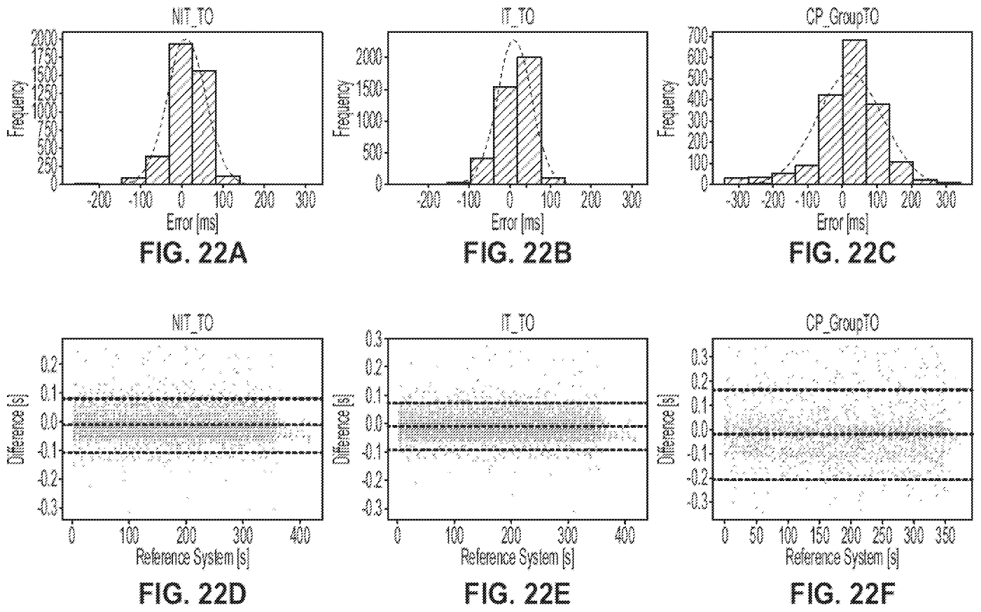

FIGS. 22A-22F show error distributions of the identification errors for TO with respect to the reference system. 22A, 22B, and 22C show a histogram of the error distributions for the three groups. The Bland-Altman plots showing the bounding error for TO for the NIT, IT, and CP groups are shown in 22D, 22E, and 22F.

For each of the P models created, the dimensions of the training datasets were kept constant by randomly selecting 5000 samples from each subject (2500 stance phase and 2500 swing phase samples). Using 5000 samples per subject means that for training, only 50 seconds out of the 6 minutes recorded are used. By decoupling the effects of the participants involved in the training, this cross-validation allows performance evaluation of the learning ability of the network architecture.

Two participants were selected and tested with each model (28 total for each group). The participants were divided into two categories: In-Training (IT) and Not-In-Training (NIT). NIT members are the participants left out of the training for the model tested. IT were participants, picked at random, whose step information were used during the training of a particular model. Each subject was tested two times, once as part of IT and once as part of NIT. If the classification performance of the network and error ranges are similar between groups, the model could be used with unknown participants without the need for a calibration session

The model with the highest test accuracy was used with a dataset of 7 children with CP. To assess the performance of the RNN, the HS and TO identified were compared against the walkway recording. Each event was paired using a maximum search window of 0.5 seconds to identify the corresponding step. Each event required the HS and TO to be identified. If any was missing, the event was counted as unidentified and was not used for the error calculation. The mean errors (ME) and mean absolute errors (MAE) were used to quantify the accuracy and precision of the RNN.

During the training, the 28 models achieved a mean accuracy (ME±SD) for classifying the gait phase (Eq. 1) of 91.45±0.27% for y at time t+1 (Eq. 4), and 91.03±0.21% for yₚ (Eq. 3) at time t−9 to time t on the training dataset. For the test dataset, the mean accuracy was 89.20±4.73% for y(t+1) and 89.08±4.64% for yₚ.

The model was able to identify 4138 out of 4198 steps for NIT comparison, each step a HS and TO, for 28 participants over 6 minutes of walking; this is a 98.6% identification rate. For the IT group, it identified 99.4% of the steps (4174). For the CP group, the RNN identified 1776 out of 2192 steps for the 7 participants; this is an 81.0% rate. For the NIT group, the model was able to achieve an accuracy and precision (ME±SD) of −5.9±37.1 ms for HS and 11.4±47.4 ms for TO. The IT group achieved an accuracy and precision of −8.3±23.5 ms for HS and 10.7±42.3 ms for TO. For the CP group, the model achieved 26.4±46.0 ms for HS and 21.0±94.6 ms for TO. Results showing the mean error and the RMSE are presented in Table for both healthy groups tested and for the CP group.

TABLE 3
Results by Group and Event in Milliseconds

| | NIT | | IT | | CP | |
|---|---|---|---|---|---|---|
| Event | ME ± SD | MAE ± SD | ME ± SD | MAE ± SD | ME ± SD | MAE ± SD |
| HS | −5.9 ± 37.1 | 23.9 ± 29.0 | −8.3 ± 23.5 | 16.8 ± 18.5 | 26.4 ± 46.0 | 35.2 ± 39.7 |
| TO | 11.4 ± 47.4 | 35.9 ± 32.8 | 10.7 ± 42.3 | 32.8 ± 28.7 | 21.0 ± 94.6 | 68.6 ± 68.6 |

The error histogram and the Bland-Altman plots between the RNN and the reference system for the three groups and the two events are presented in FIGS. 21A to 21F and FIGS. 22A to 22F. The Bland-Altman plots show that the performance of the NN is maintained over the complete recording.

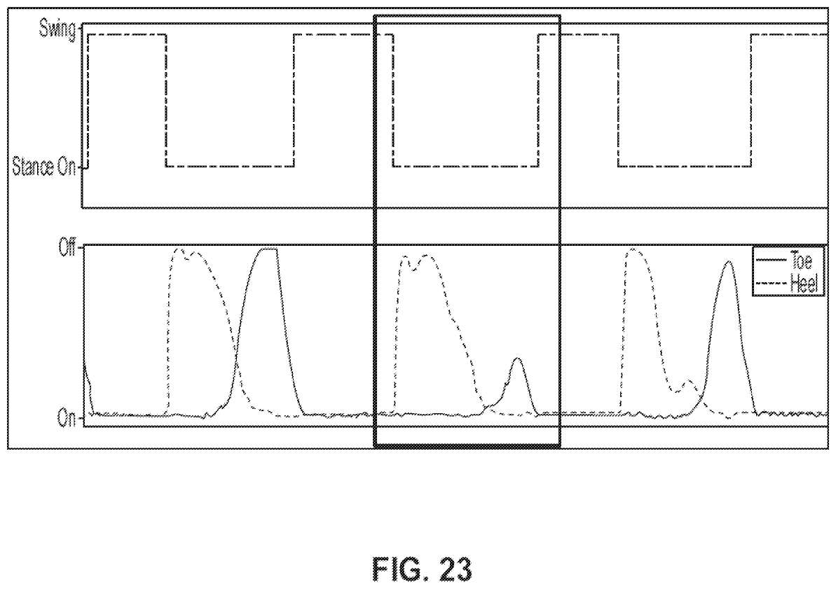

FIG. 23 shows sensor error due to variability in the walking characteristics of subjects. RNN Model can classify the data despite the misreading. Only Heel (calcaneus) and Toe (distal phalanx) are shown for clarity.

The algorithm was tested with dataset of 28 adult participants and 7 children with CP. The model was able to utilize the full range of sensors to segment the data even when sensor error was present, FIG. 23. The classification capabilities were maintained when the subject was not involved in the training. This was tested using LOOCV; the precision and accuracy were maintained between the NIT and IT groups. This means that the RNN architecture learned to classify the gait by using the multi-dimensional space created by the pressure and inertial sensors and could be used without subject specific calibration.

The results in this study show that the algorithm presented, based on RNN for segmentation and estimation of temporal parameters of gait, provides reliable performance compared to a commonly used instrumented walkway when tested with healthy adults. Furthermore, it has a similar accuracy and performance to other Machine Learning algorithms that use techniques like Hidden Markov Models or Bayesian Models, even when it was tested with over 200 minutes of walking.

Even though the RNN had a diminished accuracy and identification rate when used with children with CP, the results are encouraging. Especially, when it is considered that the RNN was trained with young adults and it had never seen data from children, let alone those with CP. Children with CP often present with gait abnormalities such as equinus and calcaneus, and in-toeing and out-toeing. This makes processing the recordings even with a reference system challenging and time consuming, since it involves manual correction. With the RNN, the processing of all 7 participants took only seconds. This means that the algorithm may be used with long recordings outside of a clinic environment, where even an 80% detection rate can still provide the overall trends of the gait. Also, it is believed that by increasing the number of participants with CP and combining the datasets between adult and children participants, models can be created that are usable on both populations.

The proposed model architecture uses the parallel nature of the neural network toolboxes and its ability to compile the model for fast execution. The processing time for the complete dataset, without any pre-processing to clean the data, was reduced from hours per subject to less than one second. This time performance boost and the portability of the instrumented shoes opens the possibility to record longer sessions outside clinical settings.

With the hardware used, the gait events in real time may be classified, at a frequency of 10-20 Hz. It has been shown that during walking, most frequencies of human movement are under 6 Hz. Thus, this processing speed would be enough to capture the kinematics and kinetics during walking. By using the properties of sequence-to-sequence mapping from raw sensor data to abstract motion characteristics, ANN could be used as a real-time sensor for human motion. This could be used by other devices, like exoskeletons, or those that provide feedback during episodic events, such as Freezing of Gait in Parkinson Disease. NN may be used to obtain spatial parameters as well as temporal parameters to remove setting and time restrictions on gait analysis.

Gait training is widely used to treat gait abnormalities. Traditional gait measurement systems are confined to instrumented laboratories. Even though gait measurements can be made in these settings, it is challenging to estimate gait parameters robustly in real-time and combine them within gait rehabilitation, especially when walking over-ground. Machine learning coupled with our labs wearable instrumented shoes allows for characterization of gait parameters in real-time. The presently disclosed subject matter includes an artificial neural network that identifies gait stance phases in real-time without requiring any processing. The algorithm has consistent performance, even when tested on novel subjects. The gait phases were correctly classified 94.17±2.97% with heel strike detection 98.73±2.00%, at an average identification time of 30.60±38.51 ms. An experiment was conducted with 10 healthy subjects whose gait data was not used in training of the algorithm. The goal was to determine if subjects could increase the stance time of their dominant leg when providing vibrotactile feedback cues. The results of the experiments showed that the subjects modified their gait in response to the feedback and walked asymmetrically. The subjects walked slower with feedback, however, retained an asymmetric gait during walking when vibration was no longer provided.

Gait training is widely used in the treatment of gait deficits. It has been shown to have positive results in different populations, such as with people who had a stroke or have knee osteoarthritis. Gait feedback can be discrete when a gait event is detected, often called open-loop, or continuous during the whole cycle based on errors from a specified trajectory, also called closed-loop. For both situations, it is vital to track gait events and phase of the gait in order to provide correct feedback during the gait cycle.

A gait cycle is defined from a Heel Strike (HS) of one foot to the next heel strike of the same foot. It consists of both stance and swing phases. Stance phase is the period when a foot is in contact with the ground, from the HS to Toe Off (TO) of the same foot (FIG. 1). Swing phase is the period when a foot is off the ground, from TO to the next HS. Stance phase is divided into initial double support, from dominant HS until TO of non-dominant leg; single support from TO of non-dominant leg until HS of non-dominant leg; and terminal double support, from HS of non-dominant leg until TO of dominant leg. Terminal double support of dominant leg is the initial double support of non-dominant leg.

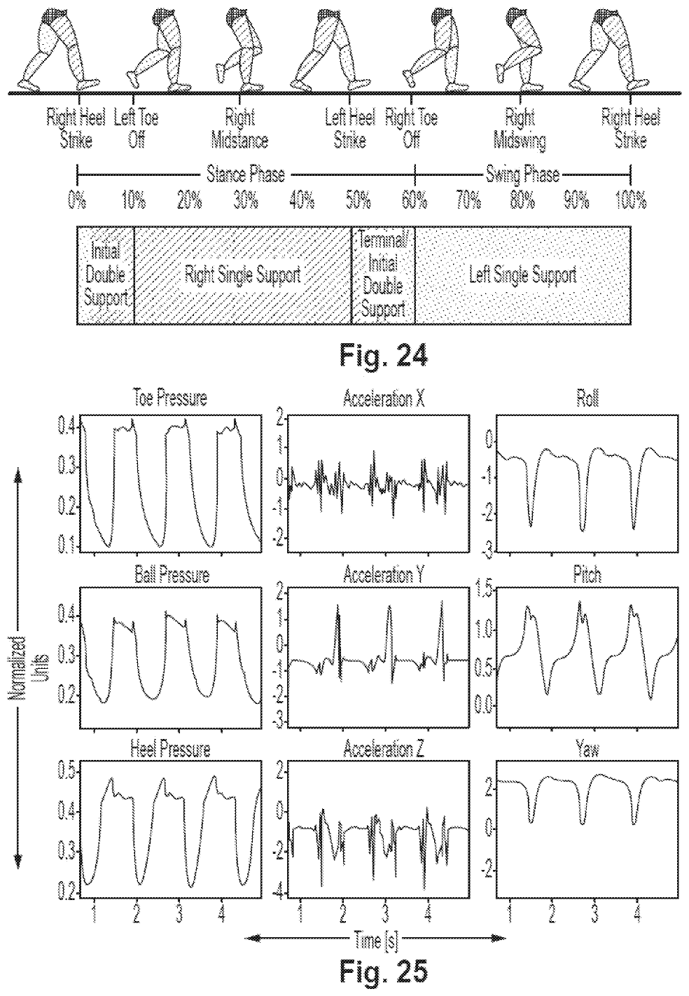

FIG. 24: shows the different phases and events in a normal gait cycle. Including gait events like Heel Strike and Toe Off, and Single and Double support stages.

HS and TO are widely used in open-loop strategies to provide feedback to the subjects based on event detection. To track the human gait in real-time, several strategies have been proposed. Most accurate strategies involve motion capture systems or instrumented mats. These devices are quite precise but are limited to constrained environments.

Wearable devices can potentially make gait analysis convenient and portable and the users are not confined to a limited area. Wearable devices can be used not only for characterization but also for long-term status monitoring. State-of-the-art wearable devices use e-Textiles and flexible electronics to conform to the shape of the user to have minimum impact in their motion. But this added flexibility increases the sensor variability and reduces the accuracy of the measurements. To maintain good levels of accuracy, wearable devices can be paired with algorithms that enhance their performance.

To identify the important gait events using wearable devices, different algorithms have been proposed which use thresholding and machine learning. These algorithms are based on identification of patterns in the sensor signals, often these patterns are called engineered features. Since this is challenging, most algorithms focus on single sensors, such as accelerometers or pressure sensors. Furthermore, the algorithms require preprocessing of the data using filters to reduce noise. Hence, a balance is needed the between number of sensors, preprocessing, and the computational load to achieve event detection and accuracy.

One way to solve this problem is by using an Artificial Neural Network (ANN). Using a training dataset, ANNs can automatically identify the patterns in the signals and map these to a desired output function. ANNs can handle multi dimensional data and can also find the interaction between the signals. This property allows the network to analyze several different signals in a single step. In order to learn complicated high-level features in multi-dimensional data, deep learning architectures have been proposed with stochastic gradient descent and backpropagation. These architectures allow ANN to calculate gradients based on loss functions, and the weights in the network are updated to optimize the performance.

FIG. 25 shows a data sample of 5 seconds collected by the DeepSole system. The first column is the pressure sensor data in volts; the lower the value, the higher the load. The second column is the acceleration in g in the IMU frame. The third column is the Euler angle rotations. FIGS. 18A, 18B and 18C shows features of the DeepSole system.

Convolution is a mathematical operator that filters data that have spatial or temporal correlations. Convolutional Neural Networks (CNN) use this operator to find the kernel parameters automatically and this reduces the noise by encoding and decoding the data. It has been successfully used to identify human motion from the signal of several Inertial Measurement Units (IMU). signal of several Inertial Measurement Units (IMU).

Recurrent Neural Networks (RNN) are used to capture time dependencies in the data. They have the ability to process information sequentially one element at a time, this gives the ability to generate sequence-to-sequence mapping. RNN models use leaky units to help the network maintain its state, accumulate data over time, and forget the previous states when they are no longer relevant.

Several algorithms have been proposed to calculate gait parameters from wearable devices. These algorithms vary in accuracy, latency, and event detection. Hence, not all of these are suitable for gait training. Karuei et al. presented an algorithm to analyze walking cadence with a smartphone at a rate of 0.5 Hz. Delgado Gonzalo et al. used accelerometer-based smart shoes to estimate gait parameters, such as the stance time (ST) at a frequency of 1 Hz. Hwang et al. proposed a head-worn device with an Inertial Measurement Unit (IMU) to detect gait events using a thresholding algorithm. However, they only validated the number of events but not the latency. The average human gait is 2 Hz, therefore these algorithms would not be able to provide real-time feedback for gait training.

Morris et al. studied a shoe-integrated sensor system for wireless gait analysis and real-time feedback. They explored signal integration methods to analyze different types of signals. Machine learning techniques such as classification, regression trees and support vector machines were used to do gait pattern classification. The gait characterization and analysis were done off-line and the data were preprocessed to reduce noise. Dehzangi et al. employed convolutional neural networks for gait recognition using accelerometers and gyroscopes. Using the CNN, they were able to successfully extract discriminating features from IMU data. Zhao et al. applied an RNN with Long Short-Term Memory (LSTM) units to extract features from gait data obtained by force-sensitive resistors in the diagnosis of neurodegenerative diseases. Both studies evaluated high-level gait patterns and showed that CNNs and RNNs have the ability to learn abstractions to describe gait from wearable sensors. Prado et al. presented a deep RNN model that maps raw signals to gait phases. Although it can provide predictions with high accuracy, the few wrong predictions within one cycle make it challenging to detect gait events with low latency.

In this application, we present an algorithm which identifies the temporal gait events in near real-time using ANN. This novel algorithm combines the filtering features of CNN with time series processing features of RNN to identify the phases of gait in real-time. We show that this model can handle the raw data from different sensors and accurately identify the gait events at an average frequency of 10 Hz. To illustrate the performance of the model, an experiment was performed with 10 healthy adults wearing the DeepSole system shown in FIG. 2. The subjects were given vibro-tactile feedback during their gait cycle to encourage them to walk asymmetrically.

### I. Algorithm Design

Identifying characteristics of the gait from raw sensor signals is a challenging problem, as these vary from person to person due to physiological differences, personal traits, and the environment. However, the general patterns in the raw sensor signals remain the same over the cycles, as shown in the sample signals collected in volts, the lower the value, the higher the load. The second column is the acceleration in g in the IMU frame. Third column is the Euler rotation angles.

A. Dataset Description

The training dataset contains walking data from 28 healthy participants, 8 females and 20 males (age 19 to 31). The participants walked for 6 minutes on a 7 meters instrumented walkway. Data was collected concurrently by the DeepSole system, FIG. 3, and by an instrumented Zeno Walkway (Protokinetics, Pa., USA).

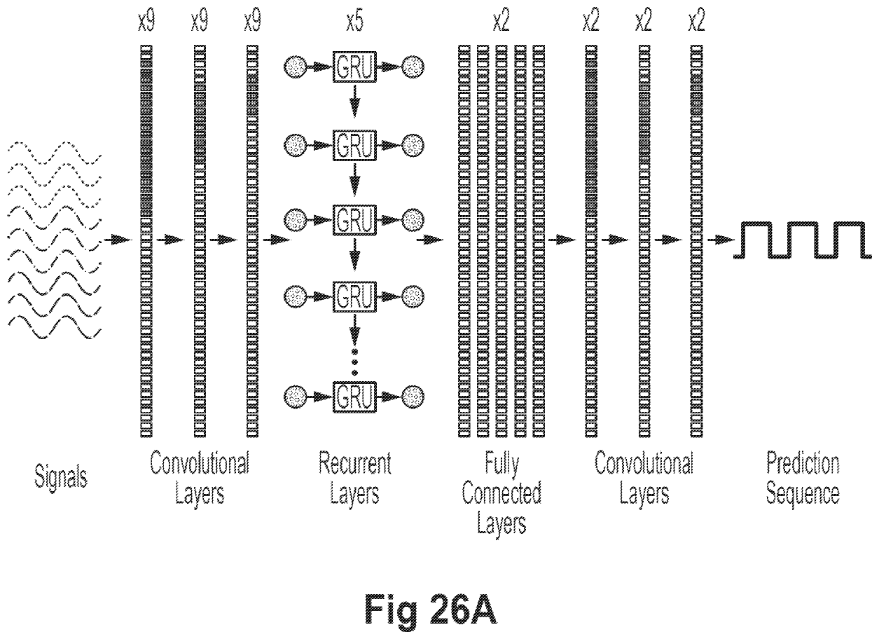

FIG. 26 shows a graphical overview of the neural network, an encoder-decoder RNN that maps the input into gait phases. The 9 signals collected by the DeepSole system are mapped to the predicted gait phase. A values of 0 corresponds to stance phase and a value of 1 corresponds to swing phase.

The DeepSole system collects signals from nine channels: Three pressure sensors, three linear accelerations, and three Euler angles. The pressure sensors are located under the phalanges, the metatarsals, and the calcaneus. The accelerations and Euler angles are measured in the local IMU coordinate system.

Signals from the shoes were collected at 200 Hz. However, in order to decrease the computational load, the training set was down-sampled to 100 Hz and the DeepSole system was modified to sample at 100 Hz during the experiment. Signals from each subject were segmented to samples of 50 continuous time points as inputs to the neural networks, i.e. 50 points at 100 Hz corresponds to moving window of 0.5 s. We assume that each sample is independent of each other, but the signal patterns are descriptive enough to be classified in the corresponding gait phase. The gait data from the Zeno Walkway were collected at 120 Hz and were used as the ground truth. From each subject, 5000 samples were randomly selected as training data.

B. Neural Network Model

The RNN presented by Prado et al. was simplified and a convolutional encoder and decoder were added to reduce the effects of noise within the network. The convolutional encoder and decoder were used to learn the temporal correlation across a time sequence. The RNN was used to learn the temporal dynamics from multi-channel time series signals. These increase the performance of the aforementioned model without increasing the computational load. The ANN maps the raw sensor signal into two classes. A value of 1 indicates swing phase and a value of 0 indicates stance phase.

Fragkiadaki et al. proposed an Encoder-Recurrent-Decoder (ERD) model to recognize and predict human body pose from video or motion capture data. The ERD was used to learn the spatial representation of human dynamics. We adopted a similar architecture in this work but we use the ERD to learn the temporal changes in the input signals.

Three convolutional layers with kernel sizes 20, 10 and 5 were used to encode signals from each channel independently. The length of the sequence was fixed throughout the convolutional layers. The convolutional output was fed into a recurrent layer with 5 Gated Recurrent Unit (GRU) cells. Dropout was used in the recurrent layers to avoid over-fitting. A fully connected layer was used to condense the recurrent outputs to a 2-class layer, and three convolutional layers with kernel sizes 20, 10 and 5 to decode the output.

A Soft-max activation was used to calculate the probability of each class and maximum likelihood was used to get the final predictions of the gait phase. The network predicted the class for all 50 time points in the input window but only the prediction for the last time point is considered a valid prediction.

FIG. 26 shows Graphical example on how each batch is created for real-time execution. The gray squares are vectors, size 9, containing the sensors measurements for one sample. The red squares are a stack of 50 vectors into a matrix of size 50×90. The blue rectangle is a tensor of 100 samples, size 100×50×9.

Cross-entropy was optimized to train the network. To address the importance of prediction for the last time point, the cross-entropy loss for the last time point was doubled. We applied stochastic gradient descent and Adam optimizer for the optimization. FIG. 4 shows a schematic of the architecture described above.

C. Online Heel Strike Detection Algorithm

Sensor readings from DeepSole system were collected at 100 Hz and stacked to create batches of data. Each batch contains 100 samples of size 50 by 9. To create these batches, the signals from the last 149 time points were used as shown in FIG. 5. For example, for the sample time t, we stack the signal from all 9 sensors from t-50 until t into a matrix of size 50 by 9. This matrix corresponds to 1 sample and this process is repeated 100 times.

Using parallel computation, 100 samples were run concurrently. The highest consistent computation frequency with the hardware used was 10 Hz. The output is the predicted gait phase for the 100 samples used. Since the model computes the prediction of 100 samples (1 seconds of recording) in parallel at an average of 10 Hz, the resulting signal can be reconstructed to the original 100 Hz without any interpolation.

To identify the HS, two moving windows were used on the output signal. One window is 30 samples long (LW) and the second window is 15 samples long (SW). HS event is labeled as identified when the average of LW is greater than the average of SW. By using this strategy, the identification algorithm can overcome a few false positives. For example, if 2 predictions were misidentified, the HS event would not be wrongly detected as the average of LW would still be greater than the average of SW. This principle is shown graphically in FIG. 6.

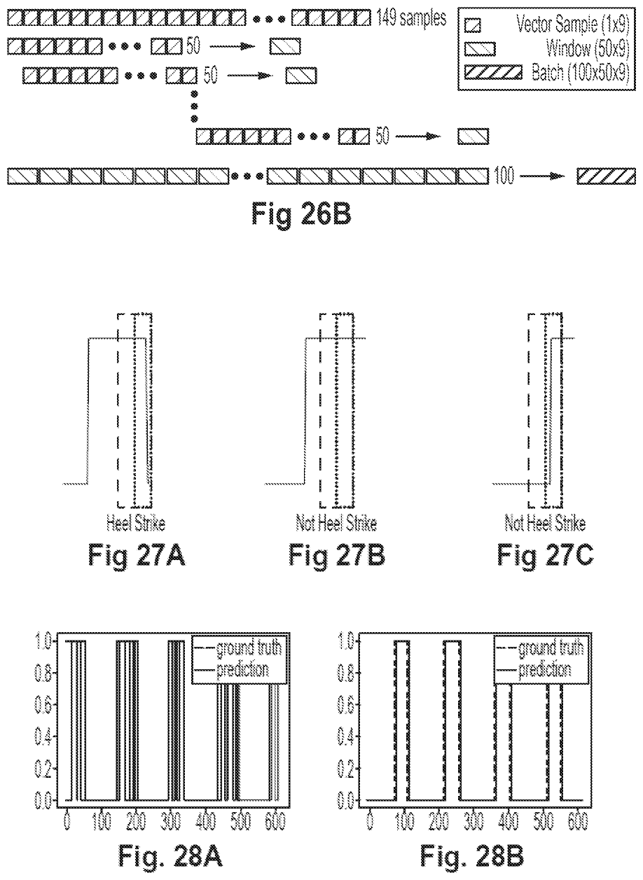

FIGS. 27A, 27B and 27C illustrate the heel strike detection algorithm. Red line is prediction of gait phase from deep model. The average of the green window and average the yellow window were compared.

To test the algorithm accuracy and precision on novel subjects, leave-one-out cross-validation test was performed over P (P=28) subjects. P models were trained using 5,000 samples from P−1 subjects. The full recording from the excluded subject was used to evaluate the performance of the model.

Convolutional encoder and decoder across time sequence is critical for real-time applications as it makes the output signal more stable. It enables continuous outputs from ANN without the need for filtering the inputs or the outputs. Without these layers, there are fluctuations in the output. As illustrated in FIG. 7(a) and FIG. 7(b), if the CNN is replaced by fully-connected layers, the output predictions are less stable, with several wrong predictions per cycle. Three parameters were tested:
1) Artificial Neural Network Accuracy: For every sample point, the model provides a prediction for the current gait phase. This output was compared to the same output measured from the reference system. The accuracy of the prediction was defined as the number of correct predictions divided by the total number of samples. The average correct prediction of the 28 models, one model for each subject, was 94.17±2.97%. This was tested with approximately 10 million samples of walking recorded by the DeepSole system for the 28 subjects.
2) Identification Rate: A HS event was labeled as correctly identified if the change of the phase was detected once in a gait cycle, else it was considered as not identified. HS identification data was calculated as the number of correct HS events out of the total number of heel strikes detected by the reference system. The average HS identification for the 28 models was 98.73±2.00%.
3) Detection Delay: Detection delay was calculated as the time difference between the HS event detected by the model and the reference system. The average delay time of the 28 models was 30.60±38.51 ms. This delay exists because the algorithm can only identify the event after it occurred.
   I. Training Study with Real-Time Feedback

To evaluate the performance of the ANN on-line, An experiment was conducted with 10 healthy adults (10 male aged 21-30, right side dominant). The subjects walked wearing the DeepSole system in a hallway equipped with a Zeno Walkway for 16 minutes. Data were recorded from the two systems simultaneously. The goal of the training was to create temporal asymmetry in gait, right vs. left leg, by providing real-time feedback using vibro-tactile actuators embedded in the shoes. The presented ANN was used to detect the HS in real-time. All subjects were novel to the ANN.

FIG. 28A shows the prediction samples from deep model with fully connected encoder and decoder. FIG. 28B shows the prediction samples from deep model with convolutional encoder and decoder.

The experiment was done in three stages. The first stage was Baseline Stage (BS), where the subjects walked at a self-selected speed for 3 minutes. This recording was used to calculate the average baseline stance time (ST) for each subject. In the Second Stage (SS), the subjects walked for 10 minutes while provided timed vibrations on the dominant side. The vibration in the foot started at HS and lasted 125% of their BS average stance time. The subjects were instructed to maintain contact of the foot with the floor while the vibration was on, underneath the foot. The goal of the training was to create temporal asymmetry by increasing the stance time of the dominant leg by 25%. For the on-dominant leg, the subjects were instructed to keep their regular gait and no vibrations were provided on that side. During the Post-Training stage (PT), subjects walked for 3 minutes. No vibration was provided but the subjects were instructed to mimic the gait from SS.

The average baseline ST for all subjects was 0.80±0.07 s for the dominant side (D). For the non-dominant side (N) it was 0.81±0.07 s. For SS and PT, the baseline average ST was used to calculate the Normalized Stance Time (NST) of the dominant and non-dominant sides.

TABLE 4
Average normalized stand time and ratio per test

| | Dominant | Non- | |
|---|---|---|---|
| | (D) | Dominant(N) | Ratio(SR) |
| BS | 1.0 ± 0.05 | 1.0 ± 0.06 | 1.0 ± 0.05 |
| SS | 1.84 ± 0.57 | 1.52 ± 0.49 | 1.27 ± 0.47 |
| PT | 1.75 ± 0.38 | 1.31 ± 0.21 | 1.36 ± 0.36 |

For SS, the average ST for the dominant side was 1.45±0.22 s and 1.19±0.24 s for the non-dominant side. For PT, the average ST for the dominant side was 1.44±0.34 s and 1.04±0.12 s for non-dominant side. Stance time symmetry ratio (SR) was defined as the ratio between ST of dominant and non-dominant sides. The average SR for all subjects during BS was 1.00±0.05, NST for dominant side was 1.00±0.05, and for non-dominant side was 1.00±0.06.

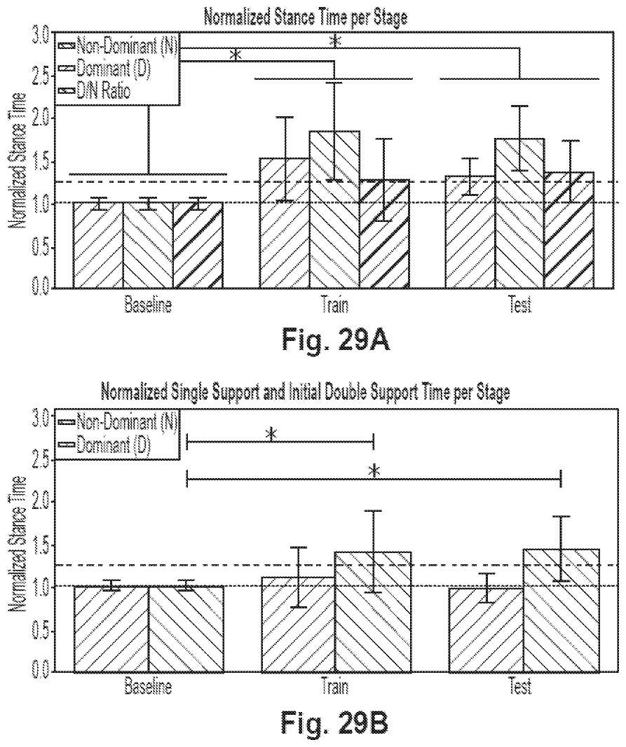

FIG. 29A show average stance time and ratio per test. Statistical significance is shown with lines. When P<0.05, * is used. The lower dotted line is the average stance time during baseline. The upper dotted line represents a 25% increase in the average stance time during baseline.

FIG. 29B shows Average Stance Time and Ratio per test. Statistical significance is shown with lines. When P<0.05, * is used. The black dotted line is the average stance time during baseline. The red dotted line represents a 25% increase in the average stance time during baseline. For SS, the average NST for dominant side of all subjects was 1.84±0.57. The average of non-dominant side was 1.52±0.49. The average SR was 1.27±0.47. During PT, the average NST for dominant side of all subjects was 1.75±0.38. The average of non-dominant side was 1.31±0.21. The average SR was 1.36±0.36. A summary of the results is shown in Table 4 and in FIG. 8. The average difference between vibration time and stance time in dominant side during SS for all subjects was 0.45±0.26 s. For PT, the average difference was 0.41±0.28 s.

A repeated measures Analysis of variance (ANOVA) was performed between experiment stages for NST of dominant and non-dominant sides and SR. Pairwise tests revealed that subjects walked significantly different during BS and SS (P<0.05), and BS and PT (P<0.05). However, there was no statistical difference between SS and PT (P=0.82). This was true for all parameters. A summary of the comparison is shown in Table 5.

TABLE 5
Values for pairwise normalized stand time and ratio per test

| | | Non- | |
|---|---|---|---|
| | Dominant(D) | Dominant(N) | Ratio(SR) |
| BS-SS | >0.01 | 0.01 | 0.047 |
| BS-PT | >0.01 | >0.01 | 0.014 |
| SS-PT | 1.00 | 0.30 | 0.62 |

The results show that subjects were able to modify their gait during training based on the haptic feedback. Furthermore, they replicated, even in the absence of feedback, as there is no significant difference between SS and PT in any of the measurements. All subjects were healthy young adults and it was expected that they would be able to follow and adapt to the feedback.

Although the ST for both dominant and non-dominant side increased, the desired 1.25 symmetry ratio is maintained for SS and PT. This result suggests that there is a delay between the desired feedback duration and the actual feedback. This delay affects both the dominant and non-dominant sides similarly, meaning the symmetry ratio is kept.

Two possible sources for the delay are the device, mainly packets of data lost through the network, and the algorithm presented, mainly the computation time. This delay is constant and could be compensated for by the system by subtracting it from the vibration time. To test the delay of the system, an evaluation was done offline with the recorded sessions. The session recordings were fed exactly as they were fed during the experiment to replicate the output signal.

The average HS identification rate during BS was 94.88±2.33% and the average detection time was 22.18±6.45 ms. For the SS, it was 89.83±2.11% with a detection time of 58.01±29.94 ms. For PT, it was 90.12±2.14% with a detection time of 45.90±13.06 ms. Since the detection time is less than computation period (100 ms), we can assume that the delay in identifying a HS event is at most 0.1 s, i.e. one computation cycle.

Even when the convolutional layers greatly decrease the number of false positives, as seen in FIG. 7, sensor noise, lost network packages, and subject variability still create false predictions and the algorithm cannot detect 100% of the events. However, an identification rate greater than 90% could still be successfully used in gait rehabilitation for several impaired populations.

The delay could also come from the human reaction time to haptic feedback. Joint movement in response to vibration has been shown to be at 0.5-1.7 Hz, but can be greatly sped up when the subjects create a memory motor trace of a cyclic movement. This means that for the subjects to start the movement after the feedback ends, it can take from 0.59 s up to 2 s if the subjects do not learn to predict the cyclic motion. During the experiment, we noticed that the subjects would hold single support during the vibration, instead of staying in terminal double support. Therefore, the subjects would only follow the vibration feedback during the initial double support and the single support time (SSDS). The terminal double support of non-dominant leg was the human reaction time to the subject.

To test this observation, a repeated measurement ANOVA of the SSDS for dominant and non-dominant sides for all stages was done. The test showed that for the non-dominant side, there was no significant difference within the three stages. But for the dominant side, there was significant difference within BS-SS and BS-PT. The value of SSDS for SS was 1.39±0.30 for dominant side and 1.06±0.26 for non-dominant side. For PT 1.45±0.97 for dominant side and 0.97±0.13 for non-dominant side. These values were normalized using the average ST during BS. FIG. 9 shows the normalized values of SSDS for all stages.

The results of the rm-ANOVA and the SSDS corroborate our observation that the subjects reaction time was contained during the terminal double support of the dominant side. The behavior of the subjects during SDSS was what we initially expected. The dominant side increased close to 125% and the non-dominant stayed close to the baseline value. This was true for all experiment stages. A summary of the comparison is shown in Table 6.

TABLE 6
P-values for normalized single support
time plus initial double support

| | Dominant(D) |
|---|---|
| BS-SS | 0.01 |
| BS-SS | 0.01 |
| SS-PT | 1.00 |

The algorithm was tested online with 10 novel subjects. Haptic feedback was provided to the subjects to increase the dominant side stance time. The subjects were able to modify their gait but there was an intrinsic delay. The delay slowed down the gait of the subjects but the desired asymmetry was maintained. The root of the delay is a reaction of the subjects to unilateral haptic feedback. During the training, the subjects waited for the feedback to stop before initiating terminal double support. This introduced a delay to the gait of 0.4 s in average, which is consistent with literature to human response time to haptic feedback.

The effect of the constant delay could be counteracted by simply shortening the duration of the desired ST time by a constant value. However, the inter-subject variability would still be present as the subjects always have a reaction time to haptic feedback. To reduce this effect, the haptic feedback could be modified from a constant vibration to a variable vibration that reduces intensity as the end of the stance phase approaches or by pairing the haptic feedback to audiovisual feedback. These strategies could help the subject create a motor memory of the desired timing, hence reducing the reaction time.

The algorithm presented is able to classify the phases of gait using the raw sensor data collected by the DeepSole system. The algorithm has consistent performance even when tested with novel subjects, maintaining over 90% classification and reconstructing the output to the original 100 Hz. Using a CNN encoder-decoder and a RNN, the system is able to map the sensors to gait phases without the need of pre-processing or post-processing. The computation can be done in real-time at an average frequency of 10 Hz with a current generation computer, but this frequency would increase as the hardware improves.

Pairing this algorithm with the DeepSole system transforms the system into a high-level sensor that provide real-time status of the gait of the user. This capability could be paired with other devices, like leg exoskeletons, to provide open-loop feedback to the user.

What is claimed is:

1. An apparatus for analyzing a subject's gait comprising:
a first foot module that includes a plurality of first pressure sensors and a first inertia measurement unit (IMU), wherein the plurality of first pressure sensors output first pressure data, and wherein the first IMU outputs a plurality of first linear accelerations, and a plurality of first Euler angles;
a second foot module that includes a plurality of second pressure sensors and a second IMU, wherein the plurality of second pressure sensors output second pressure data, and wherein the second IMU outputs a plurality of second linear accelerations, and a plurality of second Euler angles; and
an artificial neural network (ANN) configured to generate an output based on the first pressure data, the second pressure data, the plurality of first linear accelerations, the plurality of first Euler angles, the plurality of second linear accelerations, and the plurality of second Euler angles.

2. The apparatus of claim 1, wherein each of the first pressure sensors comprises a layer of piezoresistive material, and wherein each of the second pressure sensors comprises a layer of piezoresistive material.

3. The apparatus of claim 1, wherein each of the first pressure sensors comprises a layer of piezoresistive fabric positioned between two layers of conductive material, and
wherein each of the second pressure sensors comprises a layer of piezoresistive fabric positioned between two layers of conductive material.

4. The apparatus of claim 1, wherein each of the first pressure sensors comprises a layer of piezoresistive fabric positioned between two layers of conductive copper fabric, and
wherein each of the second pressure sensors comprises a layer of piezoresistive fabric positioned between two layers of conductive copper fabric.

5. The apparatus of claim 1, wherein the ANN comprises a recurrent neural network.

6. The apparatus of claim 1, wherein the ANN comprises a recurrent neural network with gated recurrent units.

7. The apparatus of claim 1, wherein the ANN comprises a recurrent neural network containing at least 8 layers, each with at least 20 gated recurrent unit cells.

8. The apparatus of claim 1, wherein the ANN comprises a recurrent neural network classifier with a plurality of classes, wherein the plurality of classes comprise a stance phase and a swing phase.

9. The apparatus of claim 1, wherein the ANN comprises a recurrent neural network, and wherein the output is a binary function of time in which one state represents stance phase and another state represents swing phase.

10. The apparatus of claim 1, wherein the ANN classifies gait events in real time at a frequency of at least 10 Hz.

11. The apparatus of claim 1, wherein the ANN combines filtering features of a convolutional neural network with time series processing features of a recurrent neural network to identify phases of the subject's gait in real time.

12. The apparatus of claim 1, wherein the ANN comprises a recurrent neural network that learns temporal dynamics from multi-channel time series signals.

13. An apparatus for analyzing a subject's gait comprising:
a recurrent neural network with gated recurrent units configured to generate an output based on (a) first pressure data, a plurality of first linear accelerations, and a plurality of first Euler angles received from a first foot module and (b) second pressure data, a plurality of second linear accelerations, and a plurality of second Euler angles received from a second foot module.

14. The apparatus of claim 13, wherein the output is a binary function of time in which one state represents stance phase and another state represents swing phase.

15. A method of analyzing a subject's gait comprising:
obtaining first pressure data, a plurality of first linear accelerations, and a plurality of first Euler angles from a first foot module positioned on a subject's left foot;
obtaining second pressure data, a plurality of second linear accelerations, and a plurality of second Euler angles from a second foot module positioned on a subject's right foot; and
processing the first pressure data, the plurality of first linear accelerations, the plurality of first Euler angles, the second pressure data, the plurality of second linear accelerations, and the plurality of second Euler angles in an artificial neural network (ANN) configured to generate an output based on the first pressure data, the second pressure data, the plurality of first linear accelerations, the plurality of first Euler angles, the plurality of second linear accelerations, and the plurality of second Euler angles.

16. The method of claim 15, wherein the ANN comprises a recurrent neural network.

17. The method of claim 15, wherein the ANN comprises a recurrent neural network classifier with a plurality of classes, wherein the plurality of classes comprise a stance phase and a swing phase.

18. The method of claim 15, wherein the ANN comprises a recurrent neural network, and wherein the output is a binary function of time in which one state represents stance phase and another state represents swing phase.

19. The method of claim 15, wherein the ANN combines filtering features of a convolutional neural network with time series processing features of a recurrent neural network to identify phases of the subject's gait in real time.

20. The method of claim 15, wherein the ANN comprises a recurrent neural network that learns temporal dynamics from multi-channel time series signals.
